(19) United States
(12) Patent Application Publication
(10) Pub. No.: US 2026/0248520 A1
(43) Pub. Date: Aug. 27, 2026

(54) PNEUMATIC SYRINGES, SUCH AS FOR CLOT ASPIRATION, AND ASSOCIATED SYSTEMS AND METHODS

(71) Applicant: Stryker Corporation, Portage, MI (US)

(72) Inventors: Daniel Aaron Leitner, Orange, CA (US); Donald Joseph Fuller, San Clemente, CA (US); Parker Ozenne, Aliso Viejo, CA (US); Jakub Marek Truty, San Clemente, CA (US); Jared Shimizu, Tustin, CA (US); Hsing-ching Crystal Hsu, Santa Ana, CA (US); Steven Samuel McConnell, Anaheim, CA (US)

(21) Appl. No.: 19/550,119

(22) Filed: Feb. 25, 2026

Related U.S. Application Data
(60) Provisional application No. 63/763,433, filed on Feb. 26, 2025.

(51) Int. Cl. A61B 17/22; A61B 17/00

(52) U.S. Cl. CPC A61B 17/22; A61B 2017/00544; A61B 2017/22079

(57) ABSTRACT

Disclosed herein are pneumatic syringes and associated systems and methods. A pneumatic syringe in accordance with the present technology can include a syringe barrel, a pneumatic barrel, and a plunger assembly extending between and movably positioned within the syringe barrel and the pneumatic barrel. The pneumatic syringe can further include a control unit configured to direct a flow of pressurized fluid from a fluid source into the pneumatic barrel to cause the plunger assembly to move between a withdrawn position and a depressed position within the syringe barrel. To move to the withdraw position, the control unit can direct the flow of pressurized fluid into a distal chamber of the pneumatic barrel to drive the plunger assembly proximally. To move to the depressed position, the control unit can direct the flow of pressurized fluid into a proximal chamber of the pneumatic barrel to drive the plunger assembly distally.

### CROSS-REFERENCE TO RELATED APPLICATION

This application claims the benefit of U.S. Provisional Patent Application No. 63/763,433, filed Feb. 26, 2025, and titled “PNEUMATIC SYRINGES, SUCH AS FOR CLOT ASPIRATION, AND ASSOCIATED SYSTEMS AND METHODS,” which is incorporated herein by reference in its entirety.

### TECHNICAL FIELD

The present technology generally relates to pneumatic syringes, such as for use in systems for treating occlusive (e.g., clot) material within a human patient, and associated systems and methods.

### BACKGROUND

Thromboembolic events are characterized by an occlusion of a blood vessel. Thromboembolic disorders, such as stroke, pulmonary embolism, heart attack, peripheral thrombosis, atherosclerosis, and the like, affect many people. These disorders are a major cause of morbidity and mortality.

When an artery is occluded by occlusive material, such as clot material, tissue ischemia develops. The ischemia will progress to tissue infarction if the occlusion persists. However, infarction does not develop or is greatly limited if the flow of blood is reestablished rapidly. Failure to reestablish blood flow can accordingly lead to the loss of limb, angina pectoris, myocardial infarction, stroke, or even death.

In the venous circulation, occlusive material can also cause serious harm. Blood clots can develop in the large veins of the legs and pelvis, a common condition known as deep venous thrombosis (DVT). DVT commonly occurs where there is a propensity for stagnated blood (e.g., long-distance air travel, immobility, etc.) and clotting (e.g., cancer; recent surgery, such as orthopedic surgery, etc.). DVT can obstruct drainage of venous blood from the legs, leading to swelling, ulcers, pain, and infection. DVT can also create a reservoir in which blood clots can collect and then travel to other parts of the body, including the heart, lungs, brain (which may cause a stroke), abdominal organs, and/or extremities.

In the pulmonary circulation, occlusive material can cause harm by obstructing pulmonary arteries—a condition known as pulmonary embolism. If the obstruction is upstream, in the main or large branch pulmonary arteries, it can severely compromise total blood flow within the lungs, and therefore the entire body, and result in low blood pressure and shock. If the obstruction is downstream, in large to medium pulmonary artery branches, it can prevent a significant portion of the lung from participating in the exchange of gases to the blood, resulting in low blood oxygen and buildup of blood carbon dioxide.

Various systems exist for performing a thrombectomy or removing occlusive material to reestablish blood flow within a patient. Such devices can remove the target clot material and often additionally remove one or more fluids (e.g., blood) along with the clot material. These devices are often designed to make it difficult to return removed fluids (e.g., blood) to the patient, even when doing so may be advantageous. For example, some existing devices include bypass circuits that can be cumbersome to set up and may require a specially trained user (e.g., a perfusionist) to operate correctly.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1 is a partially schematic side view of a clot treatment system in accordance with embodiments of the present technology.

FIG. 2 is a schematic side view of a portion of the clot treatment system of FIG. 1 in accordance with embodiments of the present technology.

FIG. 3 is a schematic side cross-sectional view of the pneumatic syringe of FIG. 2 in accordance with embodiments of the present technology.

FIGS. 4A and 4B are schematic side cross-sectional views of the pneumatic syringe of FIG. 2 in a first position and a second position, respectively, in accordance with embodiments of the present technology

FIGS. 5A and 5B are enlarged schematic side cross-sectional views of the control units of FIGS. 4A and 4B, respectively, in the first position and the second position, respectively, in accordance with embodiments of the present technology.

FIG. 6 is a partially schematic side view of a clot treatment system in accordance with embodiments of the present technology.

FIG. 7 is a partially schematic side view of a clot treatment system in accordance with additional embodiments of the present technology.

FIGS. 8A and 8B are schematic side cross-sectional views of the pneumatic syringes of FIG. 7 in a first position and a second position, respectively, in accordance with additional embodiments of the present technology.

FIGS. 9A and 9B are enlarged schematic side cross-sectional views of the remote units of FIGS. 8A and 8B, respectively, in the first position and the second position, respectively, in accordance with additional embodiments of the present technology.

FIG. 10 is a partially schematic side view of a clot treatment system in accordance with additional embodiments of the present technology.

FIG. 11 is a perspective view of a clot treatment system in accordance with additional embodiments of the present technology.

FIG. 12 is a schematic view of the clot treatment system of FIG. 11 in accordance with additional embodiments of the present technology.

FIG. 13A is a top view of a portion of the filtering device of FIG. 11 in accordance with additional embodiments of the present technology.

FIG. 13B is a top view of an interior of the filtering device of FIG. 13A in accordance with additional embodiments of the present technology.

FIG. 14 is a perspective view of the remote unit of the clot treatment system of FIG. 11 in accordance with additional embodiments of the present technology.

FIG. 15 is a flow diagram of a method for removing clot material from a patient in accordance with additional embodiments of the present technology.

### DETAILED DESCRIPTION

The present technology is generally directed to pneumatic syringes for use in clot treatment systems, and associated systems and methods. In some embodiments, the pneumatic syringe (e.g., pressure source) includes a syringe, a pneumatic cylinder, a plunger assembly, and a control unit. The plunger assembly is configured to be withdrawn through a barrel of the syringe to generate vacuum pressure. The pneumatic cylinder is coupled to the syringe and a portion of the plunger assembly. The pneumatic cylinder is fluidly coupled to a control unit configured to direct the plunger assembly from a first position to a second position and vice versa using pressurized gas from a pressurized canister when activated by a button or other actuator. In some aspects of the present technology, this configuration allows a user to hold the pneumatic syringe and withdraw the plunger assembly with one hand (e.g., by pressing the button) rather than, for example, pulling back a handle attached to the plunger assembly with one hand while the other hand holds the syringe. This can free the user to complete additional operations with their free hand, such as stabilizing a catheter. Further, automating the withdrawal and compression of the plunger assembly can result in significantly faster aspiration of the catheter and less time for the blood to be under the influence of vacuum pressure, which can lower the risk of hemolysis in aspirated blood. In other aspects of the present technology, the lack of electronic components helps minimize the risk of malfunctions within the system during procedures.

In some embodiments, the pneumatic syringe includes a syringe, a pneumatic cylinder, a plunger assembly, and a control unit with one or more ports fluidly coupled to the pneumatic cylinder. The control unit can have a first, second, and third position. In the first position, the plunger assembly is withdrawn through the syringe using pressurized gas directed through the control unit from a pressurized canister, aspirating a catheter. In the second position, the plunger assembly is depressed through the syringe using pressurized gas directed through the control unit, forcing aspirated material through a filtering device. In the third position, the control unit can block pressurized gas from entering the pneumatic cylinder to either withdraw or depress the plunger assembly. In some embodiments, the pneumatic syringe can be combined with the filtering device, reducing the complexity of the system.

In some embodiments, the pneumatic syringe includes a syringe, a pneumatic cylinder, a plunger assembly, a control unit fluidly coupled to the pneumatic cylinder, and a remote unit fluidly coupled to the control unit. Further, the system can include a catheter with a collar housing the remote unit. The remote unit can include a button actuatable by a user. The control unit can have a first and second position corresponding to the aspiration of the catheter and the filtering of aspirated material, respectively, and the remote unit can toggle the control unit between the first and second positions. In some aspects of the present technology, the collar allows the user to hold the catheter with both hands while one hand holds the collar to operate the button. This allows a user to have greater control over the placement of the catheter. In other aspects of the present technology, this configuration reduces the bulk and/or weight of the pneumatic syringe, allowing the catheter to be more easily positioned.

In some embodiments, the system includes a speed switch that allows the user to control the speed of aspiration. In some aspects of the present technology, this allows the user to refine aspiration depending on the procedure being performed. For example, slower aspiration can be performed when the catheter is positioned in delicate vasculature.

In some embodiments, the system includes an activation button and a deactivation button. When the activation button is pressed, the pressurized canister is primed, causing the control unit and/or the pneumatic cylinder to become pressurized. When the deactivation button is pressed, the control unit and/or the pneumatic cylinder are depressurized. In some embodiments, the system can include a cover that at least partially covers or obstructs the deactivation button to thereby, for example, inhibit or even prevent inadvertent actuation of the deactivation button and help ensure consistent operation of the system during aspiration and reduce the risk of wasting pressurized canisters before they are fully utilized.

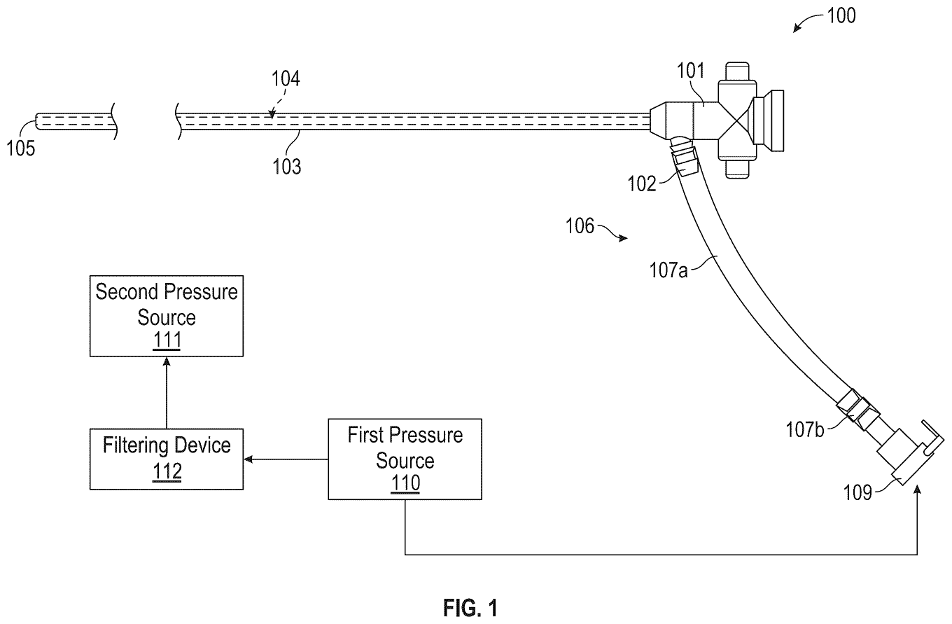

Certain details are set forth in the following description and in FIGS. 1-15 to provide a thorough understanding of various embodiments of the present technology. In other instances, well-known structures, materials, operations, and/or systems often associated with intravascular procedures, clot removal procedures, clot treatment systems, clot treatment devices, fluid control devices, syringes, blood filters, catheters, and/or the like are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth. Moreover, although many of the devices and systems are described herein in the context of removing and/or treating clot material, the present technology can be used to remove and/or treat other unwanted material in addition or alternatively to clot material, such as thrombi, emboli, plaque, intimal hyperplasia, post-thrombotic scar tissue, etc. Accordingly, the terms “clot” and “clot material” as used herein can refer to any of the foregoing materials and/or the like.

The terminology used below is to be interpreted in its broadest reasonable manner, even though it is being used in conjunction with a detailed description of certain examples of embodiments of the technology. Indeed, certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope unless expressly indicated. The sizes of various depicted elements are not necessarily drawn to scale, and these various elements may be enlarged to improve legibility. Component details may be abstracted in the Figures to exclude details such as position of components and certain precise connections between such components when such details are unnecessary for a complete understanding of how to make and use the present technology. Many of the details, dimensions, angles and other features shown in the Figures are merely illustrative of particular embodiments of the disclosure. Accordingly, other embodiments can have other details, dimensions, angles and features without departing from the present technology. In addition, those of ordinary skill in the art will appreciate that further embodiments of the present technology can be practiced without several of the details described below.

With regard to the terms “distal” and “proximal” within this description, unless otherwise specified, the terms can reference a relative position of the portions of a catheter subsystem with reference to an operator and/or a location in the vasculature. Also, as used herein, the designations “rearward,” “forward,” “upward,” “downward,” and the like are not meant to limit the referenced component to a specific orientation. It will be appreciated that such designations refer to the orientation of the referenced component as illustrated in the Figures; the systems of the present technology can be used in any orientation suitable to the user.

In the Figures, identical reference numbers identify identical, or at least generally similar, elements. To facilitate the discussion of any particular element, the most significant digit or digits of any reference number refers to the Figure in which that element is first introduced. For example, tubing assembly 106 is first introduced and discussed with reference to FIG. 1.

Descriptions of multiple positions of one or more elements should be understood to indicate that all of the elements in the system also exist in those positions accordingly. For example, if a button is in a first position, it can indicate that the other elements within the system, such as a pneumatic cylinder, are in a first position. Some elements may not change between positions while other elements may change between every position. Descriptions of elements returning from a second position to a first position can indicate that the element will be exactly as it was when it was initially in the first position or that it has changed. For example, a pressurized canister that releases gas at each position would deplete through cycling between a first and second position.

To the extent any materials incorporated herein by reference conflict with the present disclosure, the present disclosure controls.

FIG. 1 is a partially schematic side view of a clot treatment system 100 (“the system 100”) in accordance with embodiments of the present technology. The system 100 can also be referred to as an aspiration assembly, a vascular access system, a clot removal system, a thrombectomy system, and/or the like. In the illustrated embodiment, the system 100 includes a tubing assembly 106 fluidly coupled to a catheter 103 via a valve 101. In some embodiments, the catheter 103 is an elongate member (e.g., a sheath, a shaft) configured to be inserted into and through a patient's vasculature and used to, for example, remove or otherwise treat clot material therein. In other embodiments, the catheter 103 can be an introducer sheath configured to be inserted through the skin and tissue tract of the patient to provide an access site through which other components (e.g., other catheters used to treat clot material) can traverse to easily access the vasculature. Accordingly, while referred to as “catheter 103,” the catheter 103 can comprise an introducer sheath, an access sheath, and/or another type of elongate member configured to be inserted through the skin and tissue tract and/or to traverse the vasculature of a patient. The catheter 103 can be a large bore catheter, having, for example, a size equal to or greater than 16 French (Fr), such as equal to greater than 18 Fr, 20 Fr, 22 Fr, 24 Fr, 26 Fr, 28 Fr, 30 Fr, 32 Fr, and/or the like. In general, the system 100 (i) can include features generally similar in structure and/or function, or identical in structure and/or function, to those of the clot treatment systems described in detail in U.S. patent application Ser. No. 16/536,185, now U.S. Pat. No. 11,559,382, filed Aug. 8, 2019, and titled “SYSTEM FOR TREATING EMBOLISM AND ASSOCIATED DEVICES AND METHODS,” which is incorporated herein by reference in its entirety, and/or (ii) can be used to treat/remove clot material from a patient (e.g., a human patient) using any of the methods described in detail therein.

The catheter 103 further defines a lumen 104 (shown in dashed line in FIG. 1) extending entirely therethrough, e.g., from the valve 101 to a distal terminus 105 of the catheter 103. The lumen 104 is not necessarily shown to scale in FIG. 1 and can have a diameter close to the outer diameter of the catheter 103. That is the catheter 103 can have a relatively thin wall. The catheter 103 can have varying lengths, flexibilities, shapes, thicknesses, and/or other properties along its length. For example, the catheter 103 can comprise one or more coils, braids, and/or other structures positioned between one or more liner layers (e.g., an inner liner layer and an outer liner layer). In some embodiments, the catheter 103 can include several features generally similar or identical in structure and/or function to any of the catheters described in (i) U.S. patent application Ser. No. 17/529,018, titled “CATHETERS HAVING SHAPED DISTAL PORTIONS, AND ASSOCIATED SYSTEMS AND METHODS,” and filed Nov. 17, 2021, (ii) U.S. patent application Ser. No. 17/529,064, titled “CATHETERS HAVING STEERABLE DISTAL PORTIONS, AND ASSOCIATED SYSTEMS AND METHODS,” and filed Nov. 17, 2021, (iii) U.S. patent application Ser. No. 18/159,507, titled “ASPIRATION CATHETERS HAVING GROOVED INNER SURFACE, AND ASSOCIATED SYSTEM AND METHODS,” and filed Jan. 25, 2023, and/or (iv) U.S. patent application Ser. No. 18/463,960, titled “CATHETERS HAVING MULTIPLE COIL LAYERS, AND ASSOCIATED SYSTEMS AND METHODS,” and filed Sep. 8, 2023, each of which is incorporated by reference herein in its entirety.

The valve 101 is fluidly coupled to the lumen 104 of the catheter 103 and can be integral with or coupled to the catheter 103 such that these components move together. In some embodiments, the valve 101 is a hemostasis valve that is configured to maintain hemostasis during a clot treatment procedure by preventing fluid flow in a proximal direction through the valve 101 as various components such as dilators, delivery sheaths, pull members, guidewires, interventional devices, other aspiration catheters, and so on are inserted through the valve 101 to be delivered through the catheter 103 to a treatment site in a blood vessel. The valve 101 can include a branch or side port 102 configured to fluidly couple the lumen 104 of the catheter 103 to the tubing assembly 106. In some embodiments, the valve 101 can be a valve of the type disclosed in U.S. patent application Ser. No. 16/117,519, now U.S. Pat. No. 11,000,682, titled “HEMOSTASIS VALVES AND METHODS OF USE,” and filed Aug. 30, 2018, which is incorporated herein by reference in its entirety.

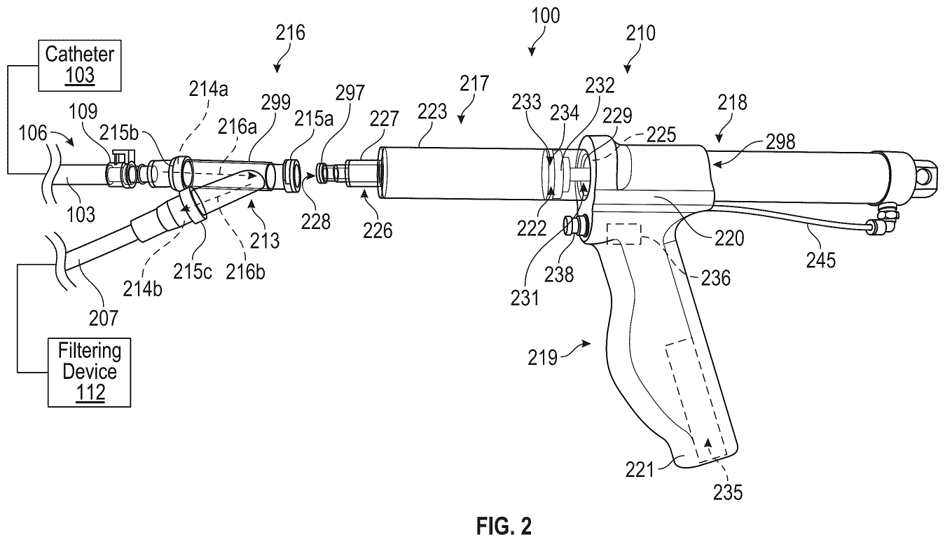

In the illustrated embodiment, the tubing assembly 106 fluidly couples the catheter 103 to a first or primary pressure source 110 (“pressure source 110”). The pressure source 110 can be a pneumatic syringe as described in detail below with reference to FIGS. 2-15. The first pressure source 110 can be configured to rapidly generate a vacuum that is applied to the lumen 104 of the catheter 103, as described in further detail below. In some embodiments, the first pressure source 110 can include some features generally similar in structure and/or function, or identical in structure and/or function, to any of the syringes described in U.S. patent application Ser. No. 16/536,185, now U.S. Pat. No. 11,559,382, filed Aug. 8, 2019, and titled “SYSTEM FOR TREATING EMBOLISM AND ASSOCIATED DEVICES AND METHODS,” which is incorporated by reference herein in its entirety. Additionally, or alternatively, the first pressure source 110 can include an electric pump and/or one or more other suitable pressure sources. In these and/or other embodiments, the first pressure source 110 can be configured to generate (e.g., form, create, charge, build-up) a vacuum (e.g., negative relative pressure) and store the vacuum for subsequent application to the catheter 103.

The tubing assembly 106 can include a first and second tubing section 107a and 107b (collectively “tubing sections 107”; which can comprise a single tube) and at least one connector 109 (e.g., a Toomey tip connector) for fluidly coupling the tubing assembly 106 to the first pressure source 110 and/or other suitable components. In some embodiments, the connector 109 is a quick-release connector (e.g., a quick disconnect fitting) that enables rapid coupling/decoupling of the catheter 103 to/from the first pressure source 110. The tubing assembly 106 and the catheter 103 can have a same or substantially same inner dimension to, for example, define a lumen or flow path of uniform or substantially uniform diameter extending from the distal terminus 105 of the catheter to the first pressure source 110.

In other embodiments, the tubing assembly 106 can further include a fluid control device (not shown) between the first tubing section 107a and the second tubing section 107b and fluidly coupled to (i) the side port 102 of the valve 101 via the first tubing section 107a and (ii) the connector 109 via the second tubing section 107b. The fluid control device can be externally operable by a user to regulate the flow of fluid therethrough and, specifically, from the lumen 104 of the catheter 103 to the first pressure source 110. For example, the fluid control device can be transitioned between (i) a first or closed configuration in which the fluid control device inhibits or even prevents fluid flow therethrough and (ii) a second or open configuration in which fluid can flow through the fluid control device. In some embodiments, the fluid control device may be closed while pressure is built in the first pressure source 110 to build vacuum pressure. The fluid control device may be opened which may create a vacuum-pressure event which may be utilized to remove clot material from a patient.

In some embodiments, the system 100 further includes a filtering device 112 and a second or secondary pressure source 111. The filtering device 112 can be configured to receive material (e.g., emboli, clot material, blood, other fluid, etc.) aspirated from the patient via the catheter 103 and to filter or otherwise separate at least a portion of the blood from this aspirated material. The second pressure source 111 can be operably coupled to (e.g., in fluid communication with) the filtering device 112 and configured to receive all, or at least a portion, of the filtered blood from the filtering device 112. Once received, the filtered blood can be reintroduced into the patient using the second pressure source 111. In some embodiments, the valve 101 and filtering device 112 can be a valve and filtering device, respectively, of the type disclosed in U.S. patent application Ser. No. 18/963,471, titled “FILTERING DEVICES, SUCH AS FOR USE WITH CLOT TREATMENT SYSTEMS, AND ASSOCIATED SYSTEMS AND METHODS,” and filed Nov. 27, 2024, which is incorporated herein by reference in its entirety.

FIG. 2 is a schematic side view of a portion of the clot treatment system 100 of FIG. 1 in accordance with embodiments of the present technology. In the illustrated embodiment, the first pressure source 110 (FIG. 1) comprises a pneumatic syringe 210 and the system 100 further includes a connector 213 positioned to fluidly couple (i) the catheter 103 to the pneumatic syringe 210 via the tubing assembly 106 and (ii) the pneumatic syringe 210 to the filtering device 112 via a tubing section 207. In some embodiments, the connector 213 can be a connector of the type disclosed in U.S. patent application Ser. No. 18/963,471, titled “FILTERING DEVICES, SUCH AS FOR USE WITH CLOT TREATMENT SYSTEMS, AND ASSOCIATED SYSTEMS AND METHODS,” and filed Nov. 27, 2024, which is incorporated herein by reference in its entirety. For example, the connector 213 can include a body 299 that defines a first through third openings or ports 215a-c (collectively “ports 215”) and a first and second flow path 216a and 216b (collectively “flow paths 216”). The body 299 can be branched, bifurcated, or y-shaped. Each of the ports 215 can be located at or proximate to a respective end or terminus of the body 299 and can be configured to be coupled (e.g., fluidly coupled) to one or more other elements in the system 100. In the illustrated embodiment, the first port 215a is coupled to the pneumatic syringe 210, the second port 215b is coupled to the catheter 103 (via, e.g., the valve 101 and the tubing assembly 106), and the third port 215c is coupled to the filtering device 112 (via, e.g., the tubing section 207).

The connector 213 can further include a first fluid control device 214a within the body 299 along the first flow path 216a (e.g., proximate the second port 215b) and a second fluid control device 214b along the second flow path 216b (e.g., proximate the third port 215c). In some embodiments, the first and second fluid control devices 214a-b can be one-way or check valves, such as a ball valve, umbrella valve, duckbill valve, cross-slit valve, dome valve, and/or the like. In other embodiments, one or both of the fluid control devices 214a-b can be active valves controlled by, for example, a user and/or an electronic control system, such as a stopcock valve, gate valve, pinch valve, and/or the like. The first fluid control device 214a is positioned to (i) permit fluid flow from the catheter 103 along the first flow path 216a to the pneumatic syringe 210 and to (ii) inhibit or even prevent (e.g., block) fluid flow from the pneumatic syringe 210 along the first flow path 216a to the catheter 103. Similarly, the second fluid control device 214b is positioned to (i) permit fluid flow from the pneumatic syringe 210 along the second flow path 216b to the filtering device 112 and to (ii) inhibit or even prevent (e.g., block) fluid from the filtering device 112 along the second flow path 216b to the pneumatic syringe 210. Accordingly, the connector 213 defines a one-way fluid flow path from the catheter 103 to the pneumatic syringe 210 and from the pneumatic syringe 210 to the filtering device 112.

In the illustrated embodiment, the pneumatic syringe 210 includes a syringe 217 and a pneumatic cylinder 218 both coupled to a handle 219. The handle 219 can include a coupling portion 220 and a grip portion 221 (e.g., a base of the handle 219). The handle 219 can define a lumen 298 extending through the coupling portion 220. The syringe 217 and the pneumatic cylinder 218 can be positioned/secured partially or completely within the lumen 298 and coupled to the coupling portion 220 of the handle 219. In the illustrated embodiment, the syringe 217 extends distally from and generally perpendicular to the handle 219 from the lumen 298 of the coupling portion 220 and the pneumatic cylinder 218 extends proximally and generally perpendicular to the handle 219 from the lumen 298 of the coupling portion 220.

In the illustrated embodiment, the syringe 217 includes a plunger assembly 222 slidably positioned within a barrel 223. The barrel 223 can be generally cylindrical and can extend between a proximal end portion 225 (obscured in FIG. 2; e.g., a proximal flange) and a distal tip 226 (e.g., a distal end portion). The proximal end portion 225 can be secured to the coupling portion 220 of the handle 219 within the lumen 298 and/or can be secured to the pneumatic cylinder 218. In some embodiments, the barrel 223 has a volume of about 60 cc or greater than about 60 cc. The distal tip 226 is configured to be releasably or permanently coupled to an adaptor 227. In some embodiments, the adaptor 227 can define a bore 228 having a size (e.g., and corresponding inner diameter) equal to or greater than 16 Fr, 18 Fr, 20 Fr, 22 Fr, 24 Fr, 26 Fr, 28 Fr, 30 Fr, 32 Fr, and/or the like. In the illustrated embodiment, the adaptor 227 is a Toomey-tip adaptor having a sealing member 297 (e.g., an O-ring) extending around an exterior surface thereof for sealingly engaging (e.g., connecting to) a Toomey fitting or Toomey adaptor, such as the first port 215a of the connector 213 (or directly to the connector 109). In other embodiments, the adaptor 227 can be omitted, and the distal tip 226 of the barrel 223 can be directly coupled to the connector 213, and/or the adaptor 227 can be another type of adaptor, such as, for example, a Luer lock, Lock slip, and/or needle.

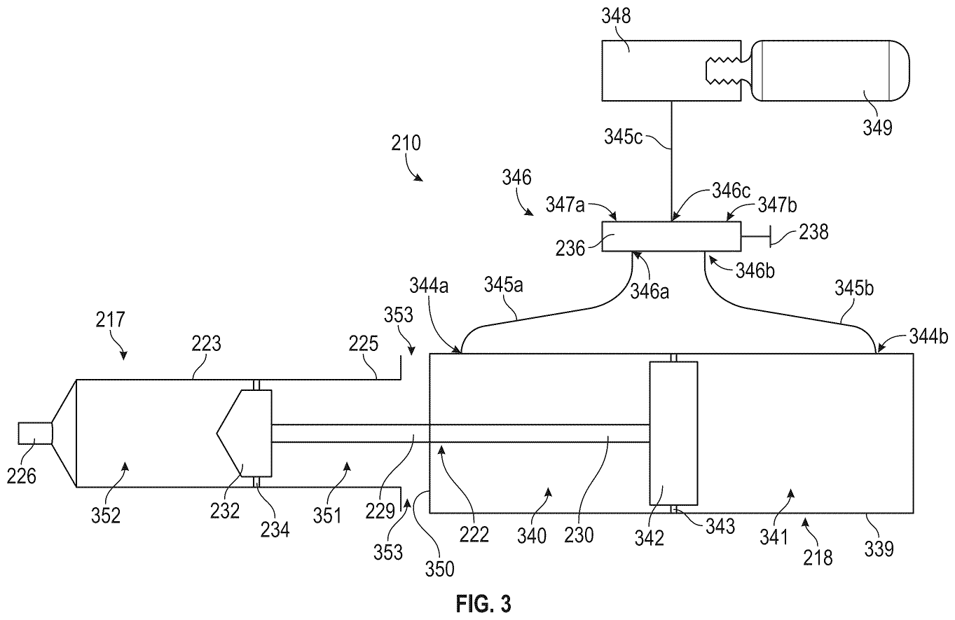

The plunger assembly 222 includes a shaft 229 (partially obscured in FIG. 2) having a proximal end portion 230 (FIG. 3) and a distal end portion 231. The distal end portion 231 of the shaft 229 can be coupled to (e.g., integrally formed, releasably, or permanently attached to) a sealing head 232. In the illustrated embodiment, the sealing head 232 has an annular shape including a circumferential groove 233. The circumferential groove 233 in the sealing head 232 can receive a sealing member 234 therein, such as an O-ring. The sealing head 232 is configured to sealingly engage an interior surface of the barrel portion 224—even as the plunger assembly 222 moves through the barrel 223—to, for example, define a sealed volume (e.g., of negative/vacuum pressure) within the barrel 223. As described in greater detail below with reference to FIG. 3, the plunger assembly 222 extends into the pneumatic cylinder 218.

The handle 219 can further define a lumen 235 (generally obscured in FIG. 2 and shown schematically) extending therethrough through the grip portion 221 toward the coupling portion 220. The lumen 235 can be shaped to accommodate a canister containing pressurized fluid (e.g., a pressurized canister; not shown, such as a carbon dioxide canister). In some embodiments, the handle 219 can include features configured to lock the pressurized canister into place once it is inserted sufficiently into the lumen 235 and to allow a user to release the pressurized canister once it is emptied or used. The handle 219 can also include additional internal channels configured to allow wires, tubes, lines, or varying mediums to pass through the handle 219. The handle 219 can be ergonomically designed so that a user can place their forefinger near the coupling portion 220 while wrapping their additional fingers along the length of the handle 219 toward the grip portion 221 to maintain a comfortable grip. In the illustrated embodiment, and as described in greater detail below, the pneumatic cylinder 218 is fluidly coupled to a control unit 236 (obscured in FIG. 2 and shown schematically) housed within the handle 219 via pressure line(s) 245 (only one shown in FIG. 2). The control unit 236 is coupled to a button 238 (e.g., switch, trigger), which is located on the handle 219, and fluidly coupled to the pressurized canister (not shown) housed within the handle 219. The button 238 can be placed so that a user can easily compress (e.g., push, activate) the button 238 with their forefinger while maintaining their grip around the handle 219. The button 238 can be configured to move between a first (e.g., compressed) and a second (e.g., released, decompressed) position based on the presence of force on the surface of the button 238. As described in further detail below, actuation of the button 238 is configured to move the plunger assembly 222 between a first (e.g., depressed) position and second (e.g., withdrawn) position, respectively. In other embodiments, the button 238 can be electrically connected to the control unit 236.

FIG. 3 is a schematic side cross-sectional view of the pneumatic syringe 210 of FIG. 2 in accordance with embodiments of the present technology. In the illustrated embodiment, the handle 219 (FIG. 2) is omitted for the sake of clarity, but it should be generally understood that any number of elements of the pneumatic syringe 210 can be housed partially or entirely within the handle 219. In the illustrated embodiment, the pneumatic cylinder 218 includes a barrel 339 having a distal end chamber 340 and a proximal end chamber 341. The barrel 339 can be coupled to and or integrally formed with the barrel 223 of the syringe 217 and fluidly separated therefrom by a shared seal wall 350. The shaft 229 of the syringe can extend through the wall 350 and into the barrel 339 of the pneumatic cylinder 218. The shaft 229 can sealingly engage an opening in the wall 350 such that the barrel 223 of the syringe 217 is fluidly disconnected from the barrel 339 of the pneumatic cylinder 218. In the illustrated embodiment, the proximal end portion 230 of the shaft 229 is movably positioned within the barrel 339 and coupled to (e.g., integrally formed, releasably or permanently attached to) a sealing head 342. In the illustrated embodiment, the sealing head 342 receives a sealing member 343, such as an O-ring in, for example, the same manner as the sealing head 232 of the syringe 217. The sealing head 342 is configured to sealingly engage an interior surface of the barrel 339—even as the plunger assembly 222 moves through the barrel 339. The sealing head 342 accordingly divides the barrel 339 into the distal (e.g., first) chamber 340 and the proximal (e.g., second) chamber 341.

In the illustrated embodiment, the sealing head 232 divides the barrel 223 into a distal chamber 352 and a proximal chamber 351 (e.g., formed between the sealing head 232 and the wall 350). The barrel 223 of the syringe 217 can include one or more vents 353 to the proximal chamber 351 at and/or proximate to the proximal end portion 225 that allow fluid (e.g., gas) to vent out of the proximal chamber 351 of the barrel 223.

The shaft 229 of the plunger assembly 222 couples the sealing head 232 of the syringe 217 to the sealing head 332 of the pneumatic cylinder 218 such that the sealing head 232 of the syringe 217 is configured to move in tandem with the sealing head 342 of the pneumatic cylinder 218, maintaining a constant distance apart. Accordingly, a location of the sealing head 342 within the barrel 339 of the pneumatic cylinder 218 determines/controls a corresponding location of the sealing head 232 within the barrel 223 of the syringe 217.

The barrel 339 further includes a first port 344a to the distal chamber 340 and a second port 344b to the proximal chamber 341. The first and second ports 344a and 344b are fluidly coupled to a first pressure line 345a and a second pressure line 345b, respectively, which are fluidly coupled to a first port 346a and a second port 346b of the control unit 236, respectively. The control unit 236 further includes a first vent 347a and a second vent 347b, which are configured to alternate allowing fluid to flow out of the barrel 339 as the pneumatic cylinder 218 cycles between a first and second position, which are described in further detail below. The first vent 347a can be smaller in size (e.g., diameter, width, bore) than the second vent 347b to allow less gas to vent out of the first vent 347a than the second vent 347b. In some embodiments, the first and second vents 347a and 347b can be the same size. In other embodiments, the first and second vents 347a and 347b can be combined into a single vent. In the illustrated embodiment, the control unit 236 further includes a third port 346c fluidly connected to a third pressure line 345c, which is fluidly coupled to a pressure regulator 348. The first, second, and third pressure lines 345a-c (collectively “pressure lines 345”) can have a same or substantially same inner dimension and can be configured to allow high-pressure gaseous or other fluid mediums to pass quickly through them. In some embodiments, the pressure lines 345 are omitted and the first and second ports 344 a and 344 b (collectively “barrel ports 344”) and first, second, and third ports 346a-c (collectively “regulator ports 346”) are directly connected to the corresponding components described above. For example, in some embodiments, the third pressure line 345c can be removed and the third port 346c can be directly connected to the pressure regulator 348.

In the illustrated embodiment, the pressure regulator 348 is fluidly coupled to a pressurized canister 349. The pressure regulator 348 can be configured to regulate the pressure from the pressurized canister 349 to ensure a constant pressure within the control unit 236. In some embodiments, the connection between the pressure regulator 348 and the pressurized canister 349 can be configured to enable a user to quickly release a depleted pressurized canister 349 and replace it with a new pressurized canister 349. The pressurized canister 349 can contain carbon dioxide (CO₂), another (e.g., high-energy density) gas, and/or a pressurized fluid. In some embodiments, the pressurized canister 349 can contain nitrogen or inert gas such as argon. In still other embodiments, the pressurized canister 349 can be replaced by a large remote tank (e.g., a tank of gas, water, or other hydraulic fluid) connected to the control unit 236 via a tubing system. In some embodiments, the pneumatic syringe 210 can include one or more additional pressure line(s) connecting the pressure regulator 348 to the pressurized canister 349. In the illustrated embodiment, the control unit 236 is configured to (i) alternate directing the pressurized gas from the pressurized canister 349 between the first port 344a and the second port 344b and (ii) to inversely alternate between allowing gas to vent out of the first vent 347a and the second vent 347b. The button 238 can be configured to switch between these alternating configurations.

In operation, fluid (e.g., gas) from the pressurized canister 349 can exert a constant pressure on the third port 346c of the control unit 236 via the third pressure line 345c. The pressure regulator 348 can regulate a pressure of the gas from the pressurized canister 349 to ensure that the pressure at the third port 346c, and accordingly the first port 344a and/or the second port 344b, does not slowly decrease as the pressurized canister 349 depletes. The control unit 236 can direct the pressurized gas to either the first port 344a or the second port 344b of the pneumatic cylinder 218 depending on whether the button 238 is in the first position (e.g., compressed, pushed) or the second position (e.g., decompressed, released), respectively. For example, when the button 238 is in the first position, the control unit 236 can direct the pressurized gas from the pressurized canister 349 into the first port 344a and the distal chamber 340 via the first pressure line 345a, filling the distal chamber 340 with gas. The pressurized gas can push the sealing head 342 of the pneumatic cylinder 218 toward the proximal end chamber 341 of the barrel 339 and, correspondingly, pull (e.g., withdraw) the sealing head 232 of the syringe 217 proximally through the barrel 223 of the syringe 217 toward the proximal end portion 225 of the barrel 223 and the pneumatic cylinder 218. Simultaneously, ambient gas (e.g., air) between the sealing head 232 and the wall 350 can vent out of the barrel 223 to atmosphere. In effect, the vents 353 inhibit or even prevent pressure from building up within the distal chamber 352 of the barrel 223 as the plunger assembly 222 moves proximally, which could otherwise create resistance against the movement of the plunger assembly 222. In some aspects of the present technology, the distal chamber 340 can be rapidly pressurized in this manner to cause rapid movement of the plunger assembly 222, nearly instantaneously generating a vacuum within the barrel 223 of the syringe 217. For example, the pneumatic force of the pressurized fluid can drive the plunger assembly 222 to fully withdraw the sealing head 232 of the syringe 217 through the barrel 223 from a depressed position to a withdrawn position in less than about 2 seconds, less than about 1 second, less than about 0.9 second, less than about 0.8 second, less than about 0.7 second, less than about 0.6 second, less than about 0.6 second, less than about 0.4 second, less than about 0.3 second, less than about 0.2 second, less than about 0.1 second, or less.

When the button 238 is in a second position (e.g., decompressed, released), the control unit 236 can direct the pressurized gas from the pressurized canister 349 into the second port 344b and the proximal chamber 341 via the second pressure line 345b, filling the proximal chamber 341 with pressurized gas. Simultaneously, the control unit 236 can connect the first port 344a with the first vent 347a via the first pressure line 345a, allowing the pressurized gas within the distal chamber 340 to vent to atmosphere. The pressurized gas within the proximal chamber 341 can push/drive the sealing head 342 of the pneumatic cylinder 218 distally through the barrel 339 toward the distal end chamber 340 of the barrel 339 and, correspondingly, the sealing head 232 of the syringe 217 toward the distal tip 226 (e.g., depressing the sealing head 232 within the barrel 223 of the syringe 217). The distal movement of the sealing head 232 within the barrel 223 can generate positive pressure in the distal chamber 352 of the syringe 217 to, for example, drive any material aspirated into the syringe 217 out of the distal tip 226. The vents 353 allow ambient gas to fill the proximal chamber 351 to inhibit or even prevent vacuum from forming in the proximal chamber 351 during distal movement of the sealing head 232. If the button 238 is pressed again (e.g., moved from the second position to the first position), the control unit 236 can again direct the pressurized gas from the pressurized canister 349 into the first port 344a via the first pressure line 345a and connect the second port 344b with the second vent 347b via the second pressure line 345b, to drive the plunger assembly 222 to retract/withdraw proximally as described in detail above.

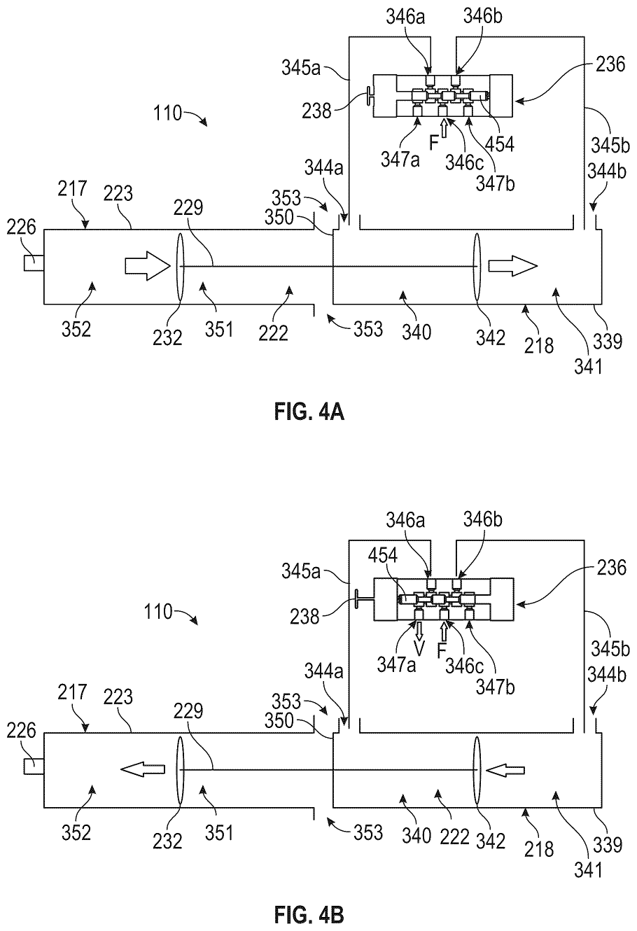

FIGS. 4A and 4B are schematic side cross-sectional views of the pneumatic syringe 210 of FIG. 2 in the first (e.g., withdrawal) position or state and the second (e.g., depression) position or state, respectively, in accordance with embodiments of the present technology. The pressure regulator 348, the pressurized canister 349, and the third pressure line 345c are omitted for the sake of clarity, but it should be understood that some or all of these components are connected to the control unit 236 via the third port 346c to supply a flow F of pressurized fluid F. In the illustrated embodiment, the control unit 236 includes a shaft 454 (e.g., an elongate member, a tube, a column) coupled to (e.g., integrally formed, releasably or permanently attached) the button 238 that is configured to control the flow F of pressurized fluid through the control unit 236. In some embodiments, the button 238 is an extension of the shaft 454 outside of the control unit 236. In other embodiments, the button 238 can be separate from the shaft 454 and electrically connected to the shaft 454.

With reference to FIG. 4A, in the withdrawal state, the button 238 is in the first position and the shaft 454 is in a corresponding first position. In the first position, the shaft 454 is configured to (i) allow fluid to pass from the third port 346c to the first port 346a through a series of chambers and (ii) allow fluid to pass from the second port 346b to the second vent 347b. Further, the shaft 454 is configured to fluidly disconnect the first vent 347a from the pneumatic cylinder 218. As such, in the withdrawal state, the flow F of pressurized fluid travels sequentially through the third port 346c, the chambers of the control unit 236, the first port 346a, the first pressure line 345a, the first port 344a, and into the distal chamber 340 of the barrel 339. The pressurized fluid fills the distal chamber 340, pushing the sealing head 342 proximally through the barrel 339 of the pneumatic cylinder 218 and thereby pulling the sealing head 232 proximally through the barrel 223 toward the wall 350. The movement of the sealing head 232 generates negative (e.g., vacuum) pressure in the distal chamber 352 of the syringe 217 that can be applied through the distal tip 226 of the syringe 217 to the aspiration catheter 103 (FIGS. 1 and 2). The negative pressure can aspirate clot material and blood through the aspiration catheter into the barrel 223. Simultaneously, in the withdrawal state, (i) fluid within the proximal chamber 341 of the pneumatic cylinder 218 vents sequentially through the second port 344b, the second pressure line 345b, the chambers of the control unit 236, and out of the vent 347b and (ii) fluid within the proximal chamber 351 of the syringe 217 vents out of the vents 353.

With reference to FIG. 4B, in the depression state, the button 238 is in a second position and the shaft 454 is in a corresponding second position. In the second position, the shaft 454 is configured to (i) allow fluid to pass from the third port 346c to the second port 346b through a series of chambers and (ii) allow fluid to pass from the first port 346a to the first vent 347a. Further, the shaft 454 is configured to fluidly disconnect the second vent 347b from the pneumatic cylinder 218. As such, in the withdrawal state, the flow F of pressurized fluid travels sequentially through the third port 346c, the chambers of the control unit 236, the second port 346b, the second pressure line 345b, the second port 344b, and into the proximal chamber 341 of the barrel 339. The pressurized fluid fills the proximal chamber 341, pushing the sealing head 342 distally through the barrel 339 of the pneumatic cylinder 218 and thereby the sealing head 232 distally through the barrel 223 of the syringe 217 toward the distal tip 226. The movement of the sealing head 232 generates positive pressure in the distal chamber 352 of the syringe 217 that can, for example, force aspirated material out of the syringe 217 through the distal tip 226. Simultaneously, (i) pressurized gas V within the distal chamber 340 of the pneumatic cylinder 218 can vent sequentially through the first port 344a, the first pressure line 345a, the first port 346a, the chambers of the control unit 236, and out of the first vent 347a and (ii) the proximal chamber 351 of the syringe 217 can remain at ambient pressure via the vents 353.

In some embodiments, the button 238 can be configured to move the shaft 454 between a first and second position with both the compression and decompression of the button 238 (e.g., the button 238 is pressed and released, corresponding to a movement of the shaft 454 into the first position and/or the button 238 is pressed and released, corresponding to a movement of the shaft 454 into the second position from the first position). For example, a user could compress the button 238 and the shaft 454 would move into the first position. When the user releases the button 238, the shaft 454 would remain in the first position until the user compresses the button 238 again, at which time the shaft 454 will move into the second position. In some embodiments, the button 238 can be configured to move the shaft 454 between a first, second, and third position with repetitive compression and/or decompression of the button 238.

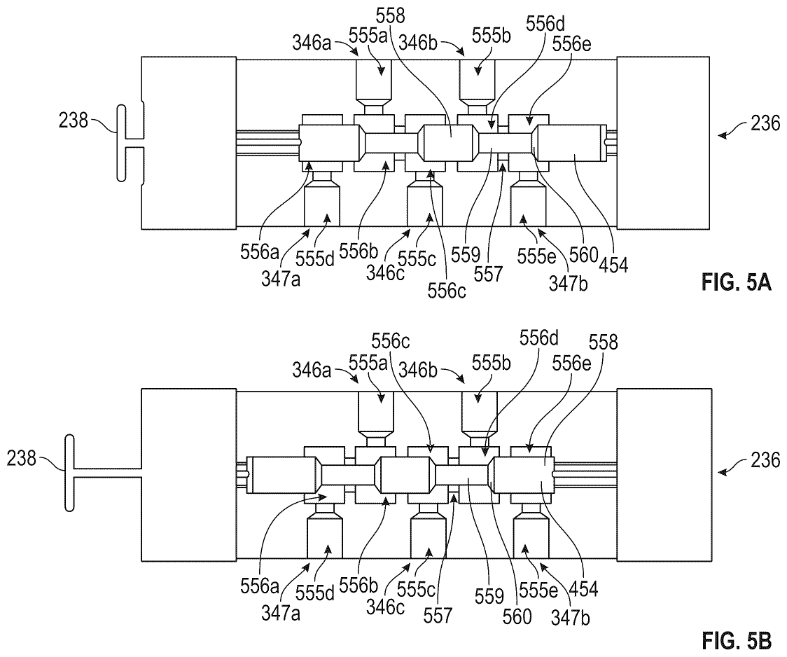

FIGS. 5A and 5B are enlarged schematic side cross-sectional views of the control unit 236 of FIGS. 4A and 4B in the withdrawal state/position and the depression state/position, respectively, in accordance with embodiments of the present technology. Referring to FIGS. 5A and 5B, in the illustrated embodiment the control unit 236 includes first through fifth introductory chambers 555a-e (collectively “introductory chambers 555”) which are fluidly connected to the first, second, and third ports 346a, 346b, and 346c, and the first and second vents 347a and 347b, respectively. The control unit 236 further includes first through fifth through chambers 556a-e (collectively “through chambers 556”). The introductory chambers 555 are fluidly coupled to the through chambers 556. Between each through chamber 556a-e and fluidly coupled to the through chambers 556 are narrow chambers 557 (not individually labeled; it should be understood there are four narrow chambers 557 in the illustrated embodiment: one between each of (i) the first and second through chambers 556a and 556b, (ii) the second and third through chambers 556b and 556c, (iii) the third and fourth through chambers 556c and 556d, and (iv) the fourth and fifth through chambers 556d and 556e).

The shaft 454 passes through each through chamber 556 and narrow chamber 557. The narrow chambers 557 are narrower than the through chambers 556 at the locations where the shaft 454 passes through each. The shaft 454 includes blocking portions 558, passing portions 559, and ramp portions 560. The ramp portions 560 extend between each of the blocking portions 558 and the passing portions 559 and the blocking portions 558 and the passing portions 559 alternate along the length of the shaft 454. The blocking portions 558 can be sized to fill the narrow chambers 557 and seal the through chamber 556 from the adjacent through chamber 556 (e.g., to inhibit or even prevent gas from passing between through chambers 556). For example, with reference to FIG. 5A, the centermost blocking portion 558 fills the narrow chamber 557, sealing the third through chamber 556c from the fourth through chamber 556d. The passing portions 559 can be sized to extend through the narrow chambers 557 without sealing the through chamber 556 from the adjacent through chamber 556 (e.g., to allow fluid to continue to pass between the through chambers 556). For example, with reference to FIG. 4A, the passing portion 559 extends through the narrow chamber 557 without sealing the fourth through chamber 556d from the fifth through chamber 556e. As such, gas can freely flow from the second introductory chamber 555b through the fourth and fifth through chambers 556d and 556e to the fifth introductory chamber 555e. The ramp portions 560 can be configured to align shaft 454 with the narrow chambers 557 as it slides through the control unit 236 between the first and second positions.

Referring to FIG. 5A, the button 238 is depressed into the first position and the shaft 454 is in the first position accordingly. In the first position, the blocking portions 558 fill the narrow chambers 557 connecting (i) the first and second through chambers 556a and 556b and (ii) the third and fourth through chambers 556c and 556d. As such, pressurized gas can pass from (i) the third introductory chamber 555c to the first introductory chamber 555a through the second and third through chambers 556b and 556c and (ii) the second introductory chamber 555b to the fifth introductory chamber 555e through the fourth and fifth through chambers 556d and 556e. The first through chamber 556a, the fourth introductory chamber 555d, and the first vent 347a are fluidly disconnected from (i) the second through fifth through chambers 556b-e and (ii) the first through third and fifth introductory chambers 555a-555c and 555e. As described in detail above with reference to FIGS. 4A and 4B, in the first position, pressurized gas passes through the third port 346c to the first port 346a and vents from the second port 346b to the second vent 347b.

Referring to FIG. 5B, the button 238 is withdrawn in a second position and the shaft 454 is in the second position accordingly. In the second position, the blocking portions 558 fill the narrow chambers 557 connecting (i) the second and third through chambers 556b and 556c and (ii) the fourth and fifth through chambers 556d and 556e. As such, pressurized gas can pass from (i) the first introductory chamber 555a to the fourth introductory chamber 555d through the first and second through chambers 556a and 556b and (ii) the third introductory chamber 555c to the second introductory chamber 555b through the third and fourth through chambers 556c and 556d. The fifth through chamber 556e, the fifth introductory chamber 555e, and the second vent 347b are fluidly disconnected from (i) the first through fourth through chambers 556a-d and (ii) the first through fourth introductory chambers 555a-d. As described in detail above with reference to FIGS. 4A and 4B, in the second position, pressurized gas passes through the third port 346c to the second port 346b and vents from the first port 346a to the first vent 347a.

In some embodiments, the button 238 has a third position. In the third position, the shaft 454 sits between the first and second positions. For example, with reference to FIG. 5A, the shaft 454 can be positioned such that the blocking portions 558 sit primarily within the first, third, and sixth through chambers 556a, 556c, and 556e while the passing portions 559 and the ramp portions 560 are positioned within the narrow chambers 557. As a result, none of the through chambers 556 would be fluidly disconnected and gas could pass throughout the control unit 236. In some aspects of the present technology, this would enable the pneumatic cylinder 218 and the control unit 236 to fully depressurize without disconnecting any of the pressure lines 345 and/or direct connections between the control unit 236 and the pneumatic cylinder 218 and/or pressurized canister 349. Further, it would enable the user to fully deplete the pressurized canister 349 before disconnecting it from the pressure regulator 348.

In other embodiments, the button 238 has an additional position that moves a blocking portion 558 of the shaft 454 to block the third introductory chamber 555c to prevent pressurized gas from the pressurized canister 349 from entering the control unit 236. For example, with reference to FIG. 5A, the shaft 454 can be positioned such that the blocking portions 558 sit primarily within the narrow chambers adjacent to the third through chamber 556c. As a result, the third introductory chamber 555c and the third through chamber 556c would be fluidly disconnected from the first, second, fourth, and fifth introductory chambers 556a, 556b, 556d, and 556e. Further, (i) the first introductory chamber 555a would be fluidly connected to the fourth introductory chamber 555d via the first and second through chamber 556a and 556b and (ii) the second introductory chamber 555b would be fluidly connected to the fifth introductory chamber 555e via the fourth and fifth through chambers 556d and 556e. More specifically, the first and second vents 347a and 347b would be unblocked and allowed to vent the pressurized gas within the pneumatic cylinder 218 and the fluidly connected portions of the control unit 236. In some aspects of the present technology, this would allow a user to depressurize the pneumatic cylinder 218 without fully depleting the pressurized canister 349.

Referring to FIGS. 3-5A, in the illustrated embodiment, the smaller size of the first vent 347a relative to the second vent 347b of the control unit 236 allows gas to vent from the vents 347 at different rates. For example, the first vent 347a can vent gas at a first rate and the second vent 347b can vent at a second rate greater than the first rate. As such, the speed at which the plunger assembly 222 retracts and depresses is different. For example, the plunger assembly 222 can retract quicker than the plunger assembly 222 can be depressed. The plunger assembly 222 can withdraw/retract more quickly as fluid in the proximal chamber 341 is quickly expelled through the larger second vent 347b without much resistance, and can depress more slowly as fluid in the distal chamber 340 is restricted to expel more slowly through the first vent 347a—increasing the resistance against the plunger assembly 222. In some aspects of the present technology, this allows a user to push the aspirated material into the filtering device 112 with less force than is used to aspirate the material, reducing the risk of damaging blood cells in the aspirated material after aspiration of the catheter 103.

In some embodiments, the pneumatic syringe 210 further comprises a switch (not shown) operably coupled to the second vent 347b of the control unit 236. The switch can be configured to move between two or more positions to control a rate at which fluid is vented through the second vent 347b from the proximal chamber 341 of the pneumatic cylinder 218. For example, the switch can be movable between (i) a first position that permits fluid to vent from the proximal chamber 341 through the second vent 347b at a first rate and (ii) a second position that permits fluid to vent from the proximal chamber 341 through the second vent 347b at a second rate less than the first rate. In such embodiments, the position of the switch can be controlled to control the vent rate through the second vent 347b to thereby control a speed at which the plunger assembly 222 retracts. For example, the plunger assembly 222 can withdraw/retract more quickly when the switch is in the first position as fluid in the proximal chamber 341 is quickly expelled through the second vent 347b without much resistance, and can withdraw/retract more slowly when the switch is in the second position as fluid in the proximal chamber 341 is restricted to expel more slowly through the second vent 347b—increasing the resistance against the plunger assembly 222. In some aspects of the present technology, the switch can be moved to the second position to, for example, inhibit or even prevent rapid aspiration of the catheter 103 (FIGS. 1 and 2) that may constrict a vessel in which the catheter 103 is inserted.

With reference to FIGS. 1 and 2, during a clot removal procedure, at least a portion of the system 100, including at least a portion of the catheter 103, can be inserted through the vasculature of a patient to treat clot material therein. In some embodiments, the system 100 is inserted to a target treatment location proximate to the clot material through an introducer sheath that traverses the skin and tissue of the patient to provide an access site. After positioning the catheter 103 at the treatment location, the pneumatic syringe 210 can be pneumatically activated (e.g., the button 238 can be pressed, the pneumatic syringe 210 can move to the first position) to rapidly generate a vacuum that is applied to lumen 104 of the catheter 103 to aspirate blood and clot material therethrough into the pneumatic syringe 210. The rapid vacuum generation can instantaneously or nearly instantaneously apply the generated vacuum pressure to the tubing assembly 106 and the catheter 103, thereby generating a suction pulse throughout the catheter 103 that can aspirate the clot material and blood into the syringe 217. More specifically, the vacuum pressure is therefore directly applied to the catheter 103 (e.g., as it is generated) to aspirate material through the tubing assembly 106, through the first fluid control device 214a, along at least a portion of the first flow path 216a, and/or into the pneumatic syringe 210. In some aspects of the present technology, the rapid movement of the pneumatically-driven plunger assembly 222 is configured to generate high flow rates—such as greater than about 40 cubic centimeters per second (CC/sec), greater than about 50 CC/sec, greater than about 60 CC/sec, greater than about 70 CC/sec, greater than about 80 CC/sec, greater than about 100 CC/sec, greater than about 120 CC/sec, greater than about 150 CC/sec, greater than about 200 CC/sec, or greater. In other aspects of the present technology, the pneumatic syringe 210 can create flow rates and pressure equalization times equal to, for example, any of the syringes described in U.S. patent application Ser. No. 16/536,185, now U.S. Pat. No. 11,559,382, filed Aug. 8, 2019, and titled “SYSTEM FOR TREATING EMBOLISM AND ASSOCIATED DEVICES AND METHODS,” which is incorporated by reference herein in its entirety, but without the use of a stopcock.

In other embodiments, the tubing assembly 106 can include a fluid control device (e.g., a stopcock) fluidly coupled to (i) the side port 102 of the valve 101 via the first tubing section 107a and (ii) the connector 109 via the second tubing section 107b, as described in detail above with reference to FIG. 1. For example, the pneumatic syringe 210 can be activated with the fluid control device in the closed position. In this manner, a vacuum is charged within the pneumatic syringe 210 (e.g., a negative pressure is maintained) before the pneumatic syringe 210 is fluidly connected to the lumen 104 of the catheter 103 (e.g., by opening the fluid control device) . To aspirate the lumen 104 of the catheter 103, the user can actuate (e.g., open) the fluid control device to fluidly connect the pneumatic syringe 210 to the catheter 103 and thereby apply or release the vacuum stored in the pneumatic syringe 210 to the lumen 104 of the catheter 103. Opening of the fluid control device instantaneously or nearly instantaneously applies the stored vacuum pressure to the tubing assembly 106 and the catheter 103, thereby generating a suction pulse throughout the catheter 103 that can aspirate the clot material and blood into the syringe 217.

In the illustrated embodiment, material aspirated via the catheter 103 can be received within the barrel 223 of the syringe 217. In these and/or other embodiments, the aspirated material can be transferred to the filtering device 112 which can filter or otherwise separate blood from the other aspirated material. For example, the pneumatic syringe 210 can be decoupled from the connector 109 and activated (e.g., by releasing the button 238, by moving the pneumatic syringe 210 to the second position) to drive blood and clot material the pneumatic syringe 210 into the filtering device 112. In other embodiments, the filtering device 112 is in line with the pneumatic syringe 210 and fluidly coupled thereto such that the pneumatic syringe 210 can transfer blood and clot material to the filtering device 112 without being decoupled from the connector 109. Once filtered by the filtering device 112, the filtered blood can be transferred to the second pressure source 111 and reinfused into the patient. In some embodiments, the second pressure source 111 can be omitted and the separated blood can be transferred to the first pressure source 110 and/or one or more other suitable pressure sources.

With reference to FIG. 2, during a clot removal procedure, the connector 213 can be configured to allow a user/operator to repeatedly aspirate material from a patient into the pneumatic syringe 210 and transfer that aspirated material to the filtering device 112, without the user/operator needing to uncouple the pneumatic syringe 210 from the catheter 103 before transferring the aspirated material to the filtering device 112. For example, during a clot removal procedure, the user activates the pneumatic syringe 210 by, e.g., pressing the button 238 to withdraw the plunger assembly 222 to aspirate clot material and blood through the catheter 103. The aspirated material can flow through the tubing assembly 106, through the first fluid control device 214a, along at least a portion of the first flow path 216a, and/or into the barrel 223. The second fluid control device 214b inhibits or even prevents aspiration through the tubing section 207 to the filtering device 112. The user can then generate a positive pressure within the pneumatic syringe 210 by, e.g., releasing the button 238 and/or depressing the plunger assembly 222 to expel the aspirated material. The expelled material is then driven along the second flow path 216b, out from the connector 213 via the third port 215c, through at least a portion of the tubing section 207, and/or into the filtering device 112. The first fluid control device 214a inhibits or even prevents the expelled material from being returned to the catheter 103. Because the fluid control devices 214 are configured to direct fluid flow through the connector 213 along the various flow paths 216 in response to the pressure generated by the pneumatic syringe 210, the user/operator can repeat the above-noted process as needed to both aspirate additional clot material from the patient and/or discharge/expel that additional clot material into the filtering device 112 without needing to uncouple the pneumatic syringe 210 from the connector 213 to, e.g., empty aspirated material from within the pneumatic syringe 210. Any clot material received within the filtering device 112 can be filtered to, e.g., remove blood at least a portion of the blood or other filtered material can be removed by the second pressure source 111.

With reference to FIGS. 1-3, the cycle (e.g., moving the button 238 between the first and second position and vice versa) can be repeated until the pressurized canister 349 is depleted. Once the pressurized canister 349 is emptied, it can be detached from the pressure regulator 348 and replaced with a new pressurized canister 349 and the cycle can resume. In some embodiments, with reference to FIG. 2, this cycle can correspond with the opening and closing of the fluid control devices 214. For example, before moving the button 238 into the first position (e.g., a position zero), the first fluid control device 214a can be opened and the second fluid control device 214b can be closed. Then, the button 238 can be compressed into the first position, aspirating the catheter 103 and pulling aspirated material along the first flow path 216a. Next, the first fluid control device 214a can be closed and the second fluid control device 214b can be opened before the button 238 is moved to the second position (e.g., while the button 238 is in the first position). Once the button 238 is moved into the second position, the aspirated material will be pushed through the second flow path 216b into the filtering device 112 via the tubing section 207. With reference to FIGS. 1 and 2, the cycle can also be completed while the fluid control devices 214 are all open, applying vacuum directly to the catheter 103.

Referring to FIGS. 1-3, in some aspects of the present technology, the pneumatic syringe 210 is configured to allow a user to drive the plunger assembly 222 to withdraw and depress with one a single hand gripping the handle 219 and a single finger of that hand actuating the button 238. This can allow the user to retain a free hand (e.g., a hand not gripping the handle 219) as the hand is not needed to, for example, pull back a handle attached to the plunger assembly 222 like in some conventional syringes. This can enable the user to complete additional operations with their free hand, such as stabilizing the catheter 103. Further, automating the withdrawal and depression of the plunger assembly 222 (e.g., movement to the first position and movement to the second position, respectively) can result in significantly faster aspiration of the catheter 103 and less time for the aspirated material to remain under vacuum pressure, which can lower the risk of hemolysis in the collected blood.

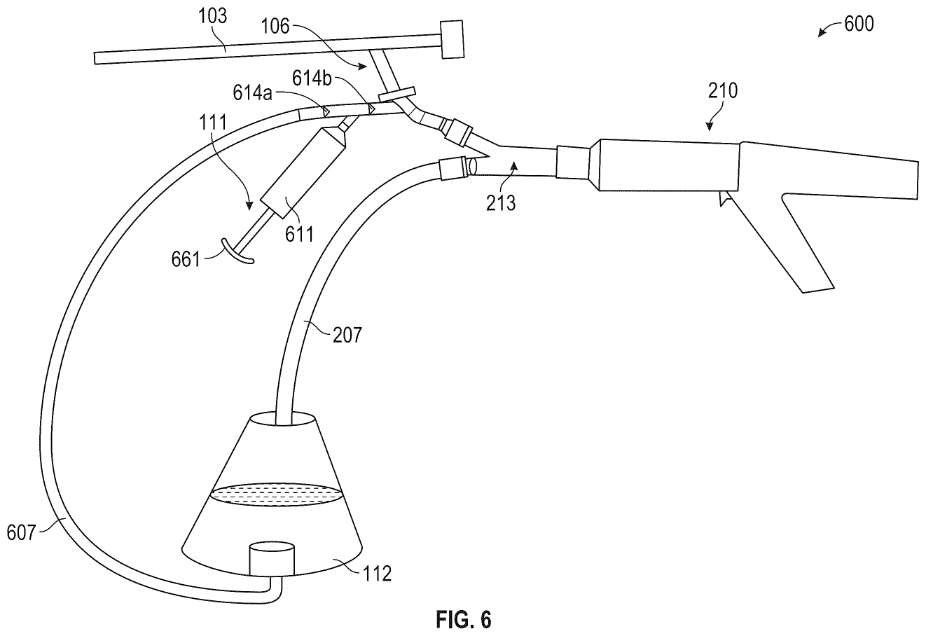

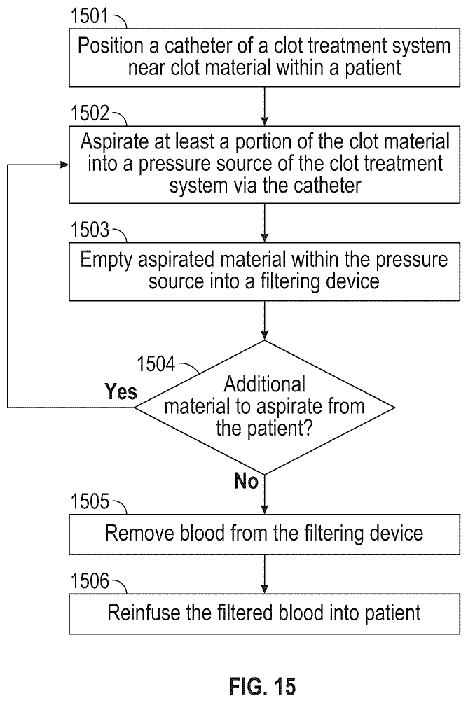

FIG. 6 is a partially schematic side view of a clot treatment system 600 in accordance with embodiments of the present technology. The system 600 can include some features that are at least generally similar in structure and function, or identical in structure and function, to the corresponding features of the system 100 as described in detail above with reference to FIG. 15B, and can operate in a generally similar or identical manner to the system 100. For example, in the illustrated embodiment the system 600 includes the catheter 103, the pneumatic syringe 210 fluidly coupled to the catheter 103 via the connector 213 and the tubing assembly 106, and the filtering device 112 fluidly coupled to the pneumatic syringe 210 via the tubing section 207 and the connector 213. In the illustrated embodiment, however, an outlet of the filtering device 112 can be fluidly coupled to the tubing assembly 106 and the catheter 103 via a tubing section 607. In the illustrated embodiment, the second pressure source 111 (FIG. 1) is a syringe 611 fluidly coupled to the tubing section 607 between a first fluid control device 614a and a second fluid control device 614b.

The syringe 611 includes a handle 661 coupled to a plunger assembly within the syringe configured to move a sealing head within the barrel of the syringe. The syringe 611 can include some features that are at least generally similar in structure and function, or identical in structure and function, to the corresponding features of the syringe 217 as described in detail above with reference to FIG. 2 and can operate in a generally similar or identical manner to the syringe 217. In the illustrated embodiment, the syringe 611 can be activated to aspirate/pull filtered blood from the filtering device 112 into the syringe 611. For example, the handle 661 can be pulled to withdraw the plunger assembly through the barrel of the syringe, creating a vacuum within the barrel of the syringe to pull blood from the filtering device 112, as described in detail above with reference to the syringe 217 of FIGS. 1-5B.

In the illustrated embodiment, the second fluid control device 614b inhibits or even prevents the vacuum pressure from being applied to the catheter 103 via the tubing assembly 106. More specially, the vacuum pressure will draw at least a portion of the blood or other filtered material through the tubing section 607, through the first fluid control device 614a, and into the syringe 611. The user can then close the first fluid control device 614a and open the second fluid control device 614b. The user can then generate a positive pressure within the syringe 611 by, e.g., depressing the plunger of the syringe to push the blood or other filter material through the tubing assembly 106 into the catheter 103. The user/operator can repeat the above-noted process as needed to return the filtered blood or other filtered material to the patient. In other embodiments, the user can open the fluid control devices 608 and repeatedly pump the syringe 611 to push the blood or other filter material through the tubing assembly 106 and into the catheter 103.

In some embodiments, the first and/or second fluid control devices 614a-b (collectively “fluid control devices 608”) can be one-way or check valves. For example, the first fluid control device 614a can allow blood and other filtered material to flow from the tubing section 607 to the syringe 611 while inhibiting or even preventing blood and other filtered material from flowing in the opposite direction from the syringe 611 to the tubing section 607. The second fluid control device 614b can allow blood and other filtered material to flow from the syringe 611 to the catheter 103 and/or the connector 213 while inhibiting or even preventing blood and other filtered material from flowing in the opposite direction from the catheter 103 and/or the connector 213 to the syringe 611. As a result, blood can generally flow through the tubing assembly 106 from the catheter 103 to the connector 213 and/or pneumatic syringe 210, through the tubing section 207, the filtering device 112, the tubing section 607, the first fluid control device 614a, and the second fluid control device 614b back to either the catheter 103 or the connector 213. In other embodiments, one or more of the fluid control devices 608 can include a ball valve, a gate valve, and/or one or more other fluid control devices (including, e.g., the fluid control devices 214 of FIG. 2) that are configured to be externally operable by a user. In some embodiments, the fluid control devices 608 can include one or more pressure-cracking valves configured to prevent blood under arterial or venous pressure within the catheter 103 from flowing into the connector 213, the pneumatic syringe 210, the syringe 611, and/or the filtering device 112 while the pneumatic syringe 210 and/or the syringe 611 are not being used.

In other embodiments, the syringe 611 can be fluidly decoupled from the tubing assembly 106 after blood and other filtered material is pulled into the syringe 611 by, e.g., withdrawing the handle 661. Further, the syringe 611 could then be fluidly coupled to the catheter 103 or other portions of the tubing assembly 106 so that the blood and other filtered material can be directly introduced into the catheter 103 or other portions of the tubing assembly 106 (or a separate reinfusion catheter) by, e.g., depressing the handle 661.

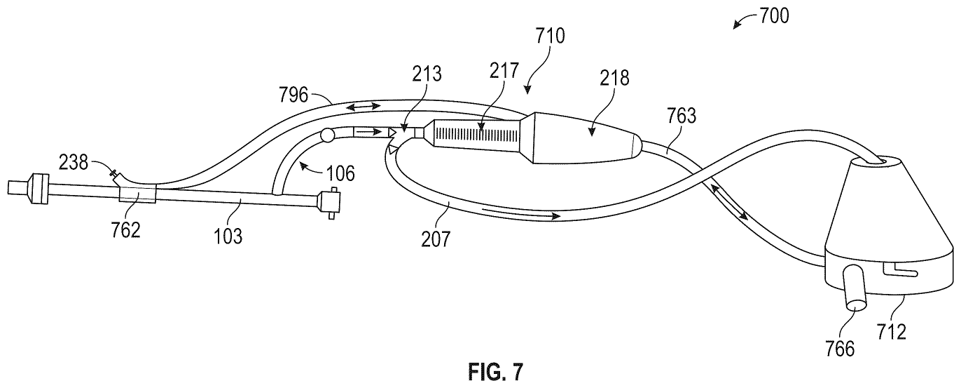

FIG. 7 is a partially schematic side view of a clot treatment system 700 in accordance with additional embodiments of the present technology. The system 700 can include some features that are at least generally similar in structure and function, or identical in structure and function, to the corresponding features of the system 100 and/or the system 600 as described in detail above with reference to FIGS. 1-6, and can operate in a generally similar or identical manner to the system 100 and/or the system 600. For example, in the illustrated embodiment the system 700 includes the catheter 103, a pneumatic syringe 710 fluidly coupled to the catheter 103 via the connector 213 and the tubing assembly 106, and a filtering device 712 fluidly coupled to the pneumatic syringe 210 via the tubing section 207 and the connector 213. The pneumatic syringe 710 includes the syringe 217 and the pneumatic cylinder 218.

In the illustrated embodiment, the catheter 103 includes a collar 762. The collar 762 surrounds the catheter 103 and is slidably coupled to the catheter 103. The collar 762 includes the button 238 fluidly coupled to a remote unit 766 via a pressure line 796. In some embodiments, the pressure line 796 can be a hydraulic hose configured to transfer force from the button 238 to the remote unit 766. The button 238 can be placed so that a user can easily compress (e.g., push, activate) the button 238 with their thumb or forefinger while maintaining their grip around the catheter 103 and/or the collar 762. In the illustrated embodiment, the remote unit 766 is housed within the filtering device 712. In some embodiments, the button 238 and the remote unit 766 are both housed in the collar 762 and directly coupled without the pressure line 796. In the illustrated embodiment, the remote unit 766 is fluidly coupled via a pressure line 763 to a control unit (not shown) generally similar to the control unit 236 described in detail above with reference to FIGS. 3-5B that controls the pneumatic syringe 710. The remote unit 766 is configured to have a first position and second position corresponding to the first and second position of the button 238 and the control unit, as described in detail above with reference to FIGS. 3-5B, respectively. For example, when the button 238 is in the first position, the remote unit 766 is in the first position and the remote unit 766 directs the control unit to the first position. Similarly, when the button 238 is in the second position, the remote unit 766 is in the second position and the remote unit 766 directs the control unit to the second position. In some aspects of the present technology, the collar 762 allows a user to hold the catheter 103 with both hands while one hand holds the collar 762 to operate the button 238. This allows a user to have greater control over the placement of the catheter 103. In other aspects, this configuration reduces the bulk/weight of the pneumatic syringe 710, allowing the catheter 103 to be more easily positioned.

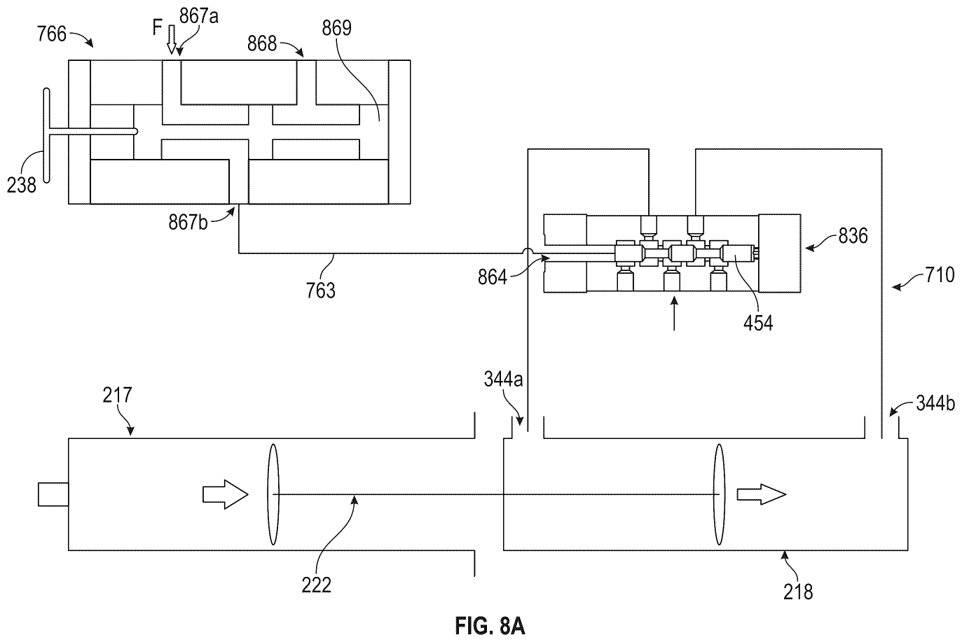

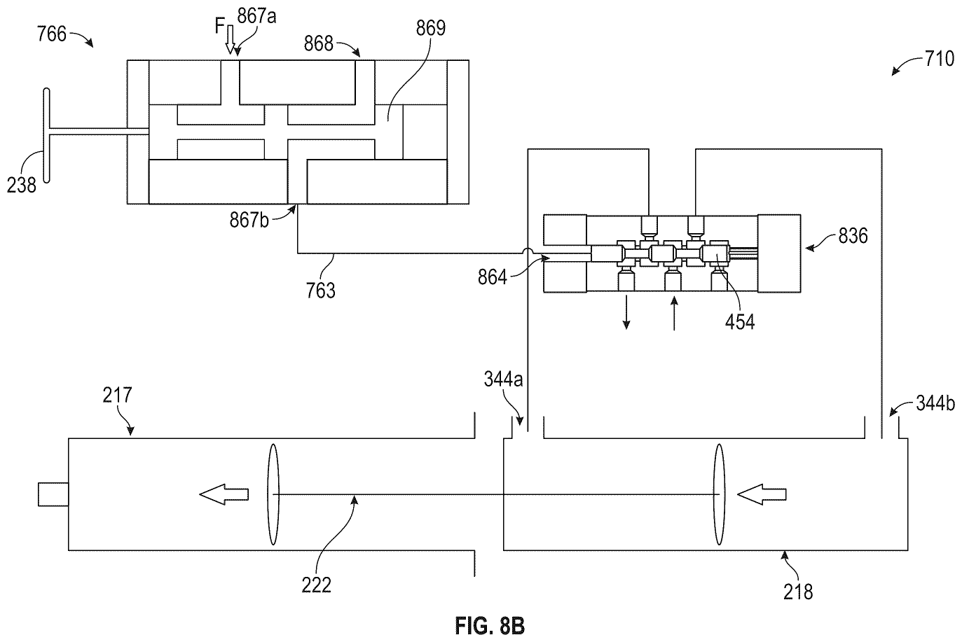

FIGS. 8A and 8B are schematic side cross-sectional views of the pneumatic syringes 710 of FIG. 7 in a first position and a second position, respectively, in accordance with additional embodiments of the present technology. The pneumatic syringe 710 can include some features that are at least generally similar in structure and function, or identical in structure and function, to the corresponding features of the pneumatic syringe 210 described in detail above with reference to FIGS. 1-6, and can operate in a generally similar or identical manner to the pneumatic syringe 210. For example, in the illustrated embodiment the pneumatic syringe 710 includes the syringe 217 and the pneumatic cylinder 218 with the plunger assembly 222. The pneumatic syringe 710 also includes a control unit 836 configured to direct the pressurized gas to the first port 344a or the second port 344b of the pneumatic cylinder 218 depending on whether the button 238 is in the first position (e.g., compressed) or the second position (e.g., released), respectively.

For the sake of clarity, in the illustrated embodiment, the pressure line 796 is omitted. In the illustrated embodiment, the pressure line 763 fluidly couples the remote unit 766 to the control unit 836 configured to control the pneumatic syringe 710. The button 238 can be configured to move between the first and the second position based on the presence of force on the surface of the button 238 and to move the control unit 836 into the first or the second position, respectively, via the remote unit 766 and the pressure line 763, as described in greater detail below. In other embodiments, the remote unit 766 and the button 238 can be located within a foot pedal at the user's feet rather than in the collar 762. In some aspects of the present technology, this would allow a user to have one hand on the catheter 103 and the other hand free to complete additional operations.

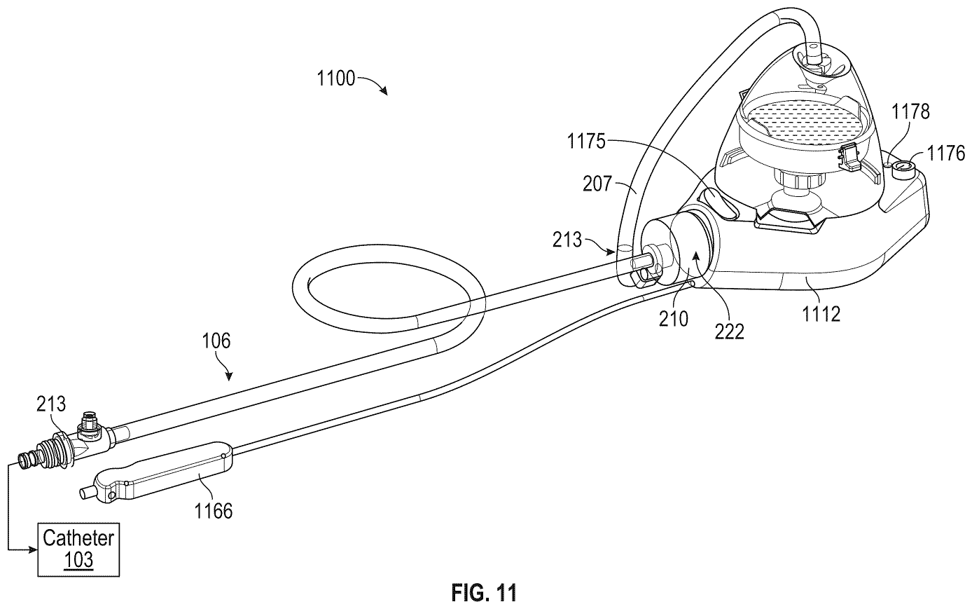

In the illustrated embodiment, the control unit 836 includes a control port 864 configured to receive pressurized gas from the remote unit 766, via the pressure line 763. When the control port 864 receives pressurized gas through the pressure line 763, the shaft 454 moves to the first position and allows gas to pass through the control unit 836 and the pneumatic cylinder 218 as described in detail above with reference to FIGS. 4A and 5A. When the control port 864 is depressurized, the shaft 454 moves to the second position and allows gas to move through the control unit 836 and the pneumatic cylinder 218 as described in detail above with reference to FIGS. 4B and 5B. In the illustrated embodiment, the remote unit includes a first port 867a, a second port 867b, a vent 868, and a shaft 869 (e.g., an elongate member, a tube, a column) coupled to (e.g., integrally formed, releasably or permanently attached to) the button 238 configured to control the direction of a pressurized gas F. With reference to FIG. 6, in some embodiments, the button 238 can be fluidly coupled to the shaft 869 via pressure line 796. In the illustrated embodiment, the first port 867a is fluidly coupled to a pressurized canister (not shown) generally similar and/or identical to the pressurized canister 349 described above with reference to FIG. 3. The pressurized canister is housed within the filtering device 712 (FIG. 6) and coupled to the remote unit 766 via a pressure line (not shown). In some embodiments, the pressurized canister is housed within the pneumatic syringe 710 or the collar 762 and coupled to the remote unit 766 either directly or via a pressure line. In other embodiments, such as system 1100 described in greater detail below with reference to FIGS. 11-14, the remote unit 766 and the control unit 836 can operate from a single pressurized canister via pressure lines and/or a pressure regulator. In the illustrated embodiment, the second port 867b is fluidly coupled to the control port 864 via the pressure line 763. In some embodiments, there is no pressure line 763 and the second port 867b is directly connected to the control port 864. In the illustrated embodiment, the pressurized canister generates the pressurized gas F, which flows through the remote unit 766 and the pressure line 763. In other embodiments, the first port 867a is fluidly coupled to a pressure regulator that includes some features that are at least generally similar in structure and function, or identical in structure and function, to the corresponding features of the pressure regulator 348 described in detail above with reference to FIG. 3 and can operate in a generally similar or identical manner to the pressure regulator 348.

With reference to FIG. 8A, the button 238 is compressed into the first position and the shaft 869 is in the first position accordingly. In the first position, the shaft 869 is configured to allow gas to pass from the first port 867a to the second port 867b through a series of chambers. Further, the vent 868 is fluidly disconnected from the rest of the remote unit 766. As such, in the first position, the pressurized gas F travels sequentially through the first port 867a, the chambers of the remote unit 766, the second port 867b, the pressure line 763, and into the control port 864. This pushes the shaft 454 into the first position and allows gas to pass through the control unit 836 and into/out of the pneumatic cylinder 218 as described in detail above with reference to FIGS. 4A and 5A. With reference to FIG. 8B, the button 238 is decompressed into a second position and the shaft 869 is in the second position accordingly. In the second position, the shaft 869 is configured to allow gas to pass through the second port 867b to the vent 868. Further, the first port 867a is fluidly disconnected from the rest of the remote unit 766 and remains at a constant positive pressure due to the pressurized gas F. As such, in the second position, the pressurized gas within the pressure line 763 and control port 864 from the pressurization while the shaft 869 was in the first position travels sequentially from the control port 864 through the second port 867b, the chambers of the remote unit 766, and out of the vent 868. This releases the shaft 454 and returns it to the second position, allowing gas to pass through the control unit 836 and into/out of the pneumatic cylinder 218 as described in detail above with reference to FIGS. 4B and 5B.

In other embodiments, with reference to FIGS. 7 and 8A, the button 238 can be electrically connected to the shaft 454 of the control unit 836 and the remote unit 766 and the pressure line 763 can be removed. For example, the button 238 could be coupled to the collar 762 and communicate with the shaft 454 of the control unit 836 electronically to direct the shaft 454 between the first and second positions.

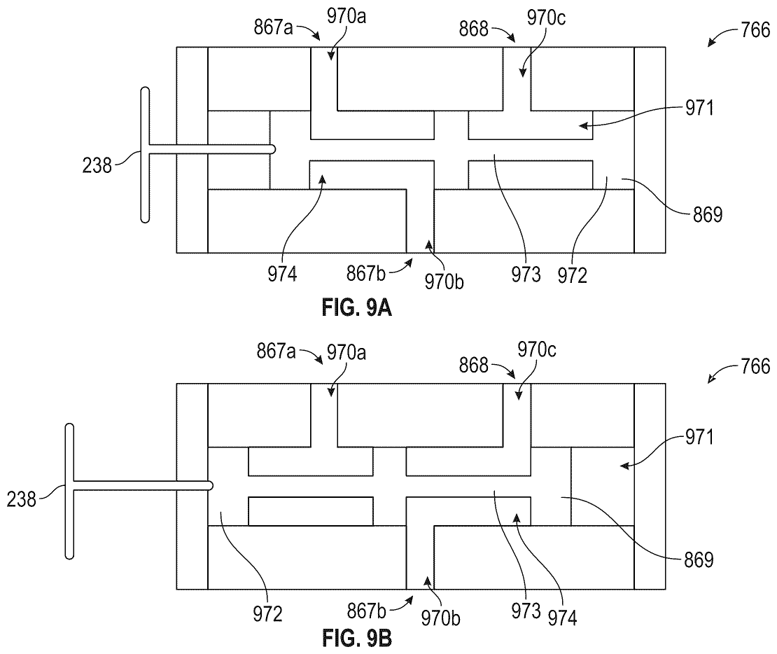

FIGS. 9A and 9B are enlarged schematic side cross-sectional views of the remote units 766 of FIGS. 8A and 8B, respectively, in the first position and the second position, respectively, in accordance with additional embodiments of the present technology. In the illustrated embodiment, the remote unit 766 includes first through third introductory chambers 970a-c (collectively “introductory chambers 970”) which are fluidly connected to the first port 867a, the second port 867b, and the vent 868, respectively. The remote unit 766 further includes a through chamber 971. The introductory chambers 970 are fluidly coupled to the through chamber 971 and the shaft 869 extends through the through chamber 971. The shaft 869 includes blocking portions 972 and passing portions 973 alternating along the length of the shaft 869. The blocking portions 972 can be sized to fill a portion of the through chamber 971 and create a seal within the through chamber 971 (e.g., to prevent gas from passing beyond the blocking portion 972). For example, with reference to FIG. 8A, the blocking portion 972 fills the through chamber 971, sealing third introductory chamber 970c from the first and second introductory chambers 970a and 970b. The passing portions 973 can be sized to extend through the through chamber 971 while allowing gas to flow through a sealed portion 974. For example, with reference to FIG. 4A, the passing portion 973 extends through the through chamber 971 without blocking gaseous flow within the sealed portion 974 of the through chamber 971. As such, gas can freely flow from the first introductory chamber 970a to the second introductory chamber 970b.

Referring to FIG. 9A, button 238 is compressed into the first position and the shaft 869 is in a first position accordingly. In the first position, the blocking portions 972 (i) fill parts of the through chamber 971 connecting the second and third introductory chambers 970b and 970c and (ii) fluidly disconnect the third introductory chamber 970c from the first and second introductory chambers 970a and 970b. As described in detail above with reference to FIG. 8A, in the first position, pressurized gas can pass from the first port 867a, through the first introductory chamber 970a, the sealed portion 974, the second introductory chamber 970b, and to the second port 867b. Referring to FIG. 9B, the button 238 is in the second position and the shaft 869 is in a second position accordingly. In the second position, the blocking portions 972 (i) fill parts of the through chamber 971 connecting the first and second introductory chambers 970a and 970b and (ii) fluidly disconnect the first introductory chamber 970a from the second and third introductory chambers 970b and 970c. As described in detail above with reference to FIG. 8B, in the second position, pressurized gas passes from the second port 867b, through the second introductory chamber 970b, the sealed portion 974, the third introductory chamber 970c, and out of the vent 868.

In some embodiments, the button 238 has a third position. In the third position, the button 238 pushes the shaft 869 to a position between the first and second positions. For example, with reference to FIG. 9A, the blocking portion 972 of the shaft 869 could be thinner (e.g., still capable of sealing the through chamber 971; thinner in the longitudinal direction) to allow the center blocking portion 972 to sit above the center of the second introductory chamber 970b while allowing gas to pass freely from both the first and third introductory chambers 970a and 970c. As a result, none of the introductory chambers 970 would be fluidly disconnected and gas could pass throughout the remote unit 766 unimpeded. In some aspects of the present technology, this would enable the remote unit 766 to fully depressurize without disconnecting the pressure line 763. Further, it would enable the user to fully deplete the pressurized canister (not shown) before disconnecting it from the remote unit 766. In some embodiments, the control unit 836 would have a corresponding third position as described in detail above with reference to FIG. 5A. In some aspects of the present technology, the remote unit 766 and/or the control unit 836 would be configured to allow their corresponding pressurized canisters to fully deplete in the third position, making it easier to change out pressurized canisters. In other embodiments, the button 238 has an additional position that moves a blocking portion 972 of the shaft 869 to block the first introductory chamber 970a to prevent pressurized gas from entering the remote unit 766 while allowing the rest of the remote unit 766 to depressurize. In some aspects of the present technology, this would allow a user to depressurize the remote unit 766 without fully depleting the pressurized canister. In further embodiments, the additional position of the button 238 could also move the shaft 454 to block the pressurized canister of the control unit 836 to prevent pressurized gas from entering the control unit 836 and/or the pneumatic cylinder 218 while allowing the rest of the control unit 836 and/or the pneumatic cylinder 218 to depressurize, as described in detail above with reference to FIG. 5A.

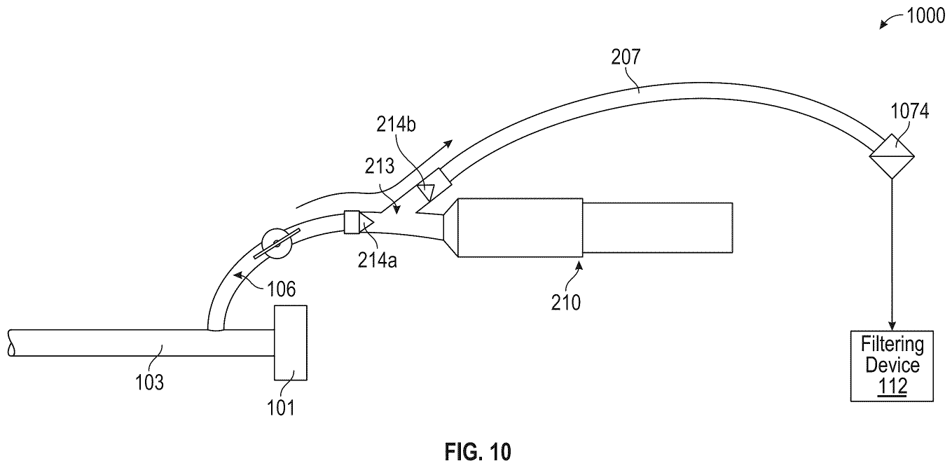

FIG. 10 is a partially schematic side view of a clot treatment system 1000 in accordance with additional embodiments of the present technology. The system 1000 can include some features that are at least generally similar in structure and function, or identical in structure and function, to the corresponding features of the system 100, the system 600, and/or the system 700 described in detail above with reference to FIGS. 1-9B, and can operate in a generally similar or identical manner to the system 100, the system 600, and/or the system 700. For example, in the illustrated embodiment, the system 1000 includes the catheter 103, the pneumatic syringe 210 fluidly coupled to the catheter 103 via the connector 213 and the tubing assembly 106, and the filtering device 112 fluidly coupled to the pneumatic syringe 210 via the tubing section 207 and the connector 213.

In the illustrated embodiment, the fluid control devices 214 are open to allow the free flow of blood and/or other material within the tubing assembly 106 and/or the connector 213 from the catheter 103. The tubing section 207 includes a pressure-cracking valve 1074 configured to prevent arterial or venous pressure from pushing blood further into the tubing assembly 106 and/or into the filtering device 112. In some embodiments, the pressure-cracking valve 1074 can be located on other tubing sections (not labeled/shown) of the tubing assembly 106. In some aspects of the present technology, this configuration is suitable for high blood pressure applications, such as arterial use, as it allows the high arterial or venous pressure to push blood into the tubing assembly 106, reducing the pressure at the distal terminus (not shown) of the catheter 103. This can reduce the pressure difference between the system 1000 and the patient's artery or vein, reducing the risk of hemolysis.

FIG. 11 is a perspective view of a clot treatment system in accordance with additional embodiments of the present technology. The system 1100 can include some features that are at least generally similar in structure and function, or identical in structure and function, to the corresponding features of the system 100, the system 600, the system 700, and/or the system 1000, as described in detail above with reference to FIGS. 1-10, and can operate in a generally similar or identical manner to the system 100, the system 600, the system 700, and/or the system 1000. For example, in the illustrated embodiment, the system 1100 includes the pneumatic syringe 210 fluidly coupled to the catheter 103 via the connector 213 and the tubing assembly 106, and a filtering device 1112 fluidly coupled to the pneumatic syringe 210 via the tubing section 207 and the connector 213.

In the illustrated embodiment, the pneumatic syringe 210 is integral with the filtering device 1112, forming a single assembly. The pneumatic syringe 210 is controlled by a remote unit 1166 operably coupled to a control unit (not shown) within the filtering device 1112. In some aspects of the present technology, combining the pneumatic syringe 210 and the filtering device 1112 can reduce the complexity of the system 1100 and/or make the system 1100 easier to transport and/or move around/position during a procedure.

In the illustrated embodiment, the filtering device 1112 includes a speed switch 1175 that can be positioned in a first position (e.g., a fast position, a fast mode) and a second position (e.g., a slow position, a slow mode) and that is operably coupled to the pneumatic syringe 210. The speed switch 1175 can be toggled (e.g., actuated) to set a speed at which the plunger assembly 222 of the pneumatic syringe 210 moves. For example, in the first position, the plunger assembly 222 can retract quickly, causing fast/rapid aspiration of the catheter 103. In the second position, the plunger assembly 222 can retract more slowly, relative to the first position, causing slower aspiration of the catheter 103. In some aspects of the present technology, the speed switch 1175 enables refined aspiration depending on the procedure being performed. For example, slower aspiration can be performed when the catheter 103 is positioned in delicate vasculature. Generally, regardless of whether the speed switch 1175 is in the first position or the second position, the pneumatic syringe 210 will aspirate the same volume of clot material through the catheter 103.

In some embodiments, the speed switch 1175 can be operably coupled to the first port 344a (FIG. 3) and/or the second port 344b (FIG. 3) of the pneumatic syringe 210 to restrict and/or allow fluid flow into the pneumatic syringe 210. For example, referring to FIGS. 3 and 11, in the first position of the speed switch 1175, the first port 344a can be unobstructed. In the second position of the speed switch 1175, the first port 344a can be partially obstructed to inhibit fluid flow out of the pneumatic syringe 210, causing the plunger assembly 222 to move more slowly relative to when the speed switch 1175 is in the first position. More specifically, actuation of the speed switch 1175 to the second position can partially restrict flow through the first port 344a to slow the exhaust of air through the first port 344a and thereby slow withdrawal of the plunger assembly 222. In some embodiments, the speed switch 1175 can be operably coupled to a fluid control device, such as the fluid control devices 214 described in greater detail above with reference to FIG. 2. In other embodiments, the speed switch 1175 can be operably coupled to the first port 346a, the second port 346b, and/or the third port 346c and/or the control port 864, the first port 867a, the second port 867b, and/or the vent 868 of FIG. 8A to control fluid flow through the system 1100. In the illustrated embodiment, the speed switch 1175 is a toggle switch. In other embodiments, the speed switch 1175 can be a different type of actuator, such as, for example, a button or a dial with one or more settings (e.g., three settings) with varied aspiration speeds.

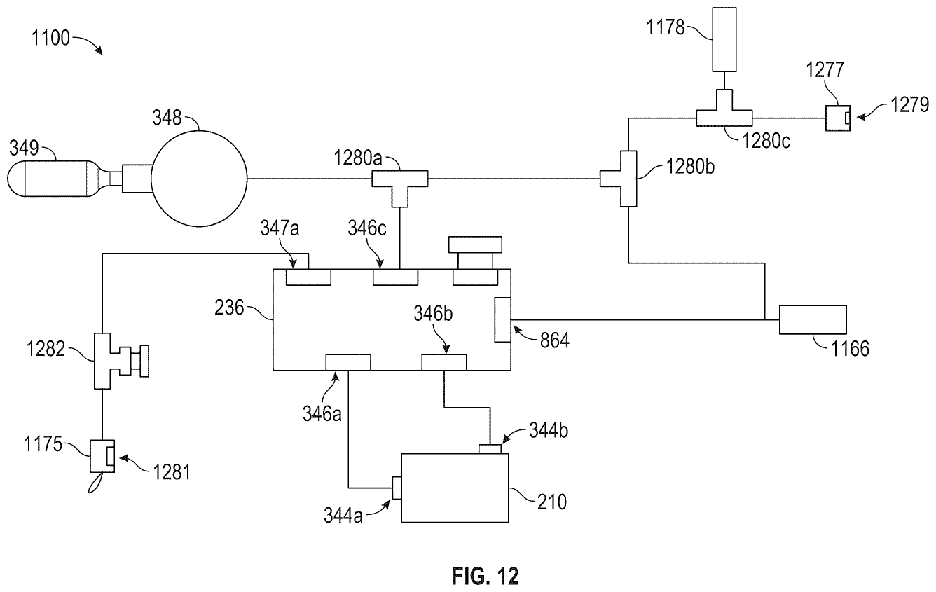

Referring again to FIG. 11, in the illustrated embodiment the filtering device 1112 further includes an activation button 1176 and a deactivation button (obscured; e.g., deactivation button 1277 (FIG. 12)). The activation button 1176 can be positioned in a first position (e.g., an uncompressed position) and a second position (e.g., a compressed position). In the illustrated embodiment, the activation button 1176 is in the first position. During operation of the system 1100, the activation button 1176 can be moved from the first position to the second position (e.g., pressed, actuated) to cause the pressurized canister 349 (FIG. 3) to be primed for aspiration. For example, referring to FIGS. 3 and 11 together, in the first position, the pressurized canister 349 can be closed such that fluid cannot flow into the pressure regulator 348 from the pressurized canister 349. Accordingly, the control unit 236 and the pneumatic syringe 210 are depressurized (e.g., at atmospheric pressure). Once the activation button 1176 is moved from the first position to the second position, the pressurized canister 349 can be opened, causing fluid to flow into the pressure regulator 348 and the control unit 236 via the third pressure line 345c, as described in greater detail above with reference to FIG. 3. For example, movement of the activation button 1176 from the first position to the second position can cause (e.g., mechanically) the pressurized canister 349 to engage a puncturing element to puncture the pressurized canister 349 and allow fluid flow into the pressure regulator 348 from the pressurized canister 349.

In the illustrated embodiment, the filtering device 1112 further includes a status indicator 1178 that indicates whether the activation button 1176 is actuated. More specifically, the status indicator 1178 has a first indication (e.g., red) and a second indication (e.g., green) that indicates to the user the status of the system 1100 (e.g., whether the pressure regulator 348 is primed). For example, before the activation button 1176 is actuated (e.g., moved to the second position), the status indicator 1178 can display the first indication. After the activation button 1176 is actuated, the status indicator 1178 can display the second indication. Accordingly, the status indicator 1178 can indicate to the user if the system 1100 is pressurized and ready for aspiration. In some embodiments, the status indicator 1178 can be a floating indicator that includes a member that moves when the system 1100 is pressurized, thus indicating to the user when the system is pressurized. In other embodiments, the status indicator 1178 includes a measuring device that determines the pressure within the system 1100, and the status indicator 1178 displays the pressure. In some embodiments, one or more of the activation button 1176, the status indicator 1178, and/or the deactivation button (e.g., deactivation button (FIG. 12)) can be incorporated into and integral with the remote unit 1166.

FIG. 12 is a schematic view of the clot treatment system 1100 of FIG. 11 in accordance with additional embodiments of the present technology. In the illustrated embodiment, the system 1100 includes the pressurized canister 349 fluidly coupled to the pressure regulator 348, which is fluidly coupled to the third port 346c of the control unit 236. The pressure regulator 348 is also fluidly coupled to the status indicator 1178 and the remote unit 1166. The status indicator 1178 is fluidly coupled to a deactivation button 1277, which can include an outlet 1279 through which fluid can exit the system 1100. The remote unit 1166 is fluidly coupled to the control port 864 of the control unit 236. The speed switch 1175 is fluidly coupled to the first vent 347a of the control unit 236. Finally, the first port 344a of the pneumatic syringe 210 is fluidly coupled to the first port 346a of the control unit 236, and the second port 344b of the pneumatic syringe 210 is fluidly coupled to the second port 346b of the control unit 236, as described in greater detail above with reference to FIG. 3.

In the illustrated embodiment, the system 1100 includes first through third connectors 1280a-1280c (collectively “connectors”) that couple various components of the system 1100 together. The connectors 1280 can also be referred to as three-way connectors, valves, adaptors, and/or fluid control devices. In the illustrated embodiment, (i) the first connector 1280a fluidly couples the pressurized canister 349, the third port 346c of the control unit 236, and the second connector 1280b; (ii) the second connector 1280b fluidly couples the first connector 1280a, the remote unit 1166, and the third connector 1280c; and (iii) the third connector 1280c fluidly couples the second connector 1280b, the status indicator 1178, and the deactivation button 1277. In other embodiments, one or more of the connectors 1280 can be omitted, and in some embodiments, additional connectors 1280 can be included to connect other components of the system 1100.

In the illustrated embodiment, the speed switch 1175 includes an outlet 1281 that allows fluid to flow out of the system 1100. The system 1100 also includes a fluid control device 1282 fluidly coupling the speed switch 1175 to the first vent 347a. The speed switch 1175 is operably coupled to the fluid control device 1282, allowing the user to control fluid flow out of the first vent 347a and out of the system 1100 via the outlet 1281. For example, when the speed switch 1175 is in the first position, the fluid control device 1282 can be fully opened to allow unobstructed fluid flow between the first vent 347a and the outlet 1281. In the second position, the fluid control device 1282 can be closed or partially closed to obstruct fluid flow between the first vent 347a and the outlet 1281, causing the plunger assembly 222 (FIG. 2) of the pneumatic syringe 210 to move more slowly. In some embodiments, one or more additional fluid control devices can be included in the system 1100 to modulate fluid flow through the system 1100, such as the fluid control devices 214 described in greater detail above with reference to FIG. 2. In some embodiments, the fluid control device 1282 can be incorporated into the speed switch 1175. For example, an open area (e.g., a cross-sectional area) of the outlet 1281 can change between the first position and the second position of the speed switch 1175.

In some embodiments, the deactivation button 1277 can include a first position (e.g., an uncompressed position) and a second position (e.g., a compressed position). During operation of the system 1100, the deactivation button 1277 can be moved from the first position to the second position (e.g., pressed, actuated) to depressurize the system 1100. For example, referring to FIGS. 11 and 12 together, the pressurized canister 349 can be primed by the activation button 1176 while the deactivation button 1277 is in the first position, allowing for the operation of the system 1100 (e.g., aspiration). Then, the deactivation button 1277 can be moved to the second position, opening the outlet 1279 and allowing fluid to escape the system 1100 through the outlet 1279. Specifically, residual fluid in the pressurized canister 349, the control unit 236, and/or the pneumatic syringe 210 could flow through the connectors 1280 to the deactivation button 1277 and out of the outlet 1279 (e.g., via the third port 346c). Accordingly, the control unit 236 and the pneumatic syringe 210 can be depressurized (e.g., to atmospheric pressure) by actuating the deactivation button 1277, causing the system 1100 to become inoperable (e.g., incapable of aspiration). For example, at the end of a clot treatment procedure, the user can press the deactivation button 1277 to inhibit, or even prevent, inadvertent aspiration of the catheter 103 (FIG. 1) as the catheter 103 is withdrawn from the patient. Likewise, the deactivation button 1277 can fully expel fluid from and depressurize the pressurized canister 349 to, for example, facilitate disposal thereof.

Referring to FIGS. 11 and 12 together, during operation of the system 1100, the remote unit 1166, the speed switch 1175, the activation button 1176, and/or the deactivation button 1277 can be operated by the user to control the operation of the system 1100. For example, prior to operation of the system 1100, the user can actuate the activation button 1176 to prime the pressurized canister 349 and pressurize the control unit 236 and the pneumatic syringe 210. Then, the user can set the speed of aspiration using the speed switch 1175. Alternatively, the speed can be set prior to priming the pressurized canister 349. Once the user is ready to aspirate the catheter 103, the user can actuate the remote unit 1166 (e.g., press the button 238 (FIG. 9A)), causing the plunger assembly 222 to be withdrawn proximally to thereby aspirate the catheter 103. The user can continue to execute aspiration cycles via the remote unit 1166, for example, as described in greater detail above with reference to FIGS. 8A and 8B, until the clot material is fully aspirated. Once the clot material is fully aspirated through the catheter 103 and pushed into or through the filtering device 1112, the user can actuate the deactivation button 1277 to depressurize the system 1100, inhibiting additional aspiration and facilitating disposal of the pressurized canister 349. After deactivation, the catheter 103 can be removed from the patient with a reduced risk of inadvertent aspiration.

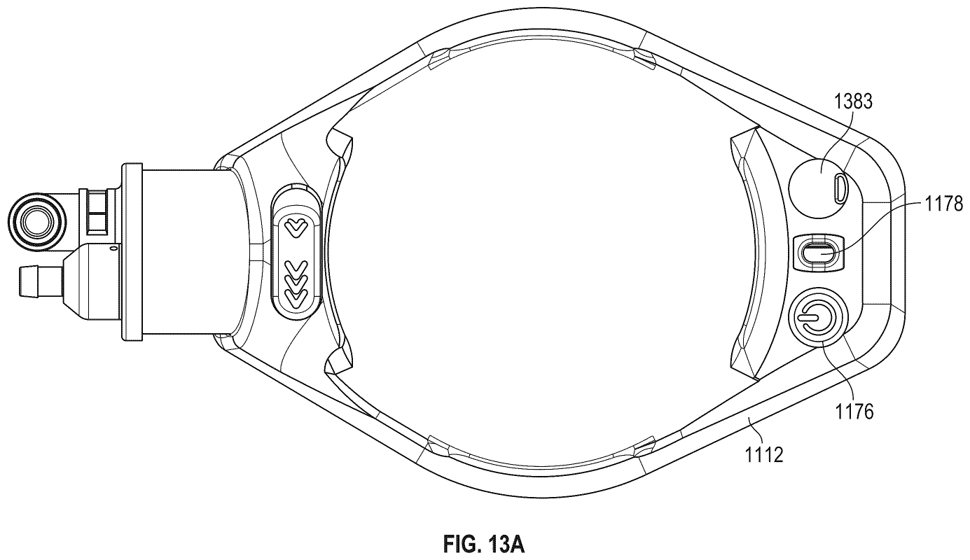

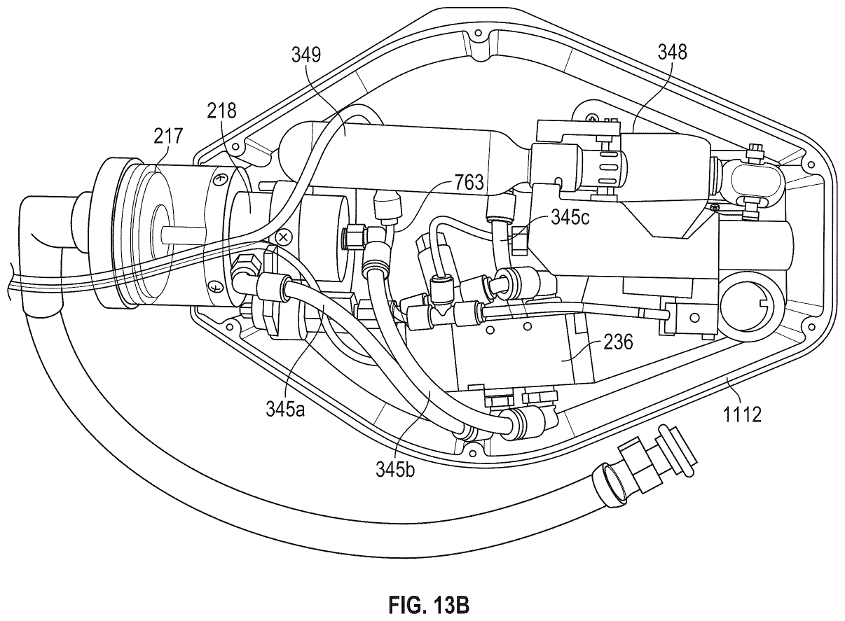

FIG. 13A is a top view of a portion of the filtering device 1112 of FIG. 11 in accordance with additional embodiments of the present technology. Specifically, a filter housing of the filtering device 1112 is omitted. In the illustrated embodiment, the filtering device 1112 includes the activation button 1176, the status indicator 1178, and the deactivation button 1277 (obscured), as described above. In the illustrated embodiment, the filtering device 1112 includes a cover 1383 that covers the deactivation button 1277, inhibiting actuation of the deactivation button 1277. The cover 1383 shown is a sliding door that can be slid forward to reveal the deactivation button 1277; however, in other embodiments, the cover 1383 can be a flip cover, a rotating cover, a screen, and/or the like. In some embodiments, the cover 1383 can be omitted, or the cover 1383 can be another type of mechanism that inhibits inadvertent actuation of the deactivation button 1277, such as a biasing member or a child-lock mechanism. In some aspects of the present technology, including the cover 1383 reduces the risk of inadvertent actuation of the deactivation button 1277, which can help ensure consistent operation of the system 1100 and reduce the risk of wasting pressurized canisters 349 (FIG. 3) before they are fully utilized.

FIG. 13B is a top view of an interior of the filtering device 1112 of FIG. 13A in accordance with additional embodiments of the present technology. In the illustrated embodiment, the filtering device 1112 houses the control unit 236, the pneumatic cylinder 218, a portion of the syringe 217, the pressurized canister 349, the pressure regulator 348, the pressure lines 345, and the pressure line 763. In other embodiments, the filtering device 1112 can include only some of these components, and, in further embodiments, the filtering device 1112 can include additional components of the system 1100, such as the fluid control device 1282 and the connectors 1280.

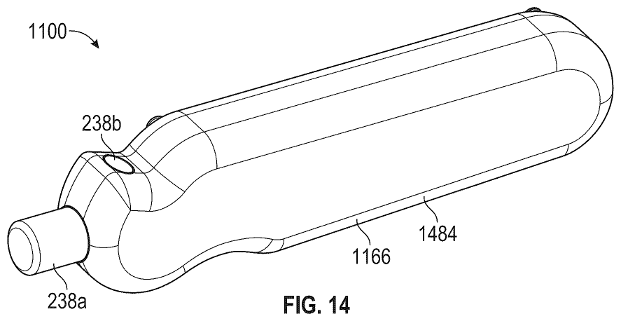

FIG. 14 is a perspective view of the remote unit 1166 of the clot treatment system 1100 of FIG. 11 in accordance with additional embodiments of the present technology. In the illustrated embodiment, the remote unit 1166 includes two buttons 238, individually labeled a first button 238a and a second button 238b, operably coupled together. The remote unit 1166 includes two positions: a first position (e.g., an aspiration position) and a second position (e.g., a reinfusion position). In the illustrated embodiment, the remote unit 1166 is in the first position. In the first position, the first button 238a extends out of a housing 1484 of the remote unit 1166 for easy actuation, and the second button 238b is positioned substantially within or fully within the housing 1484, such that the second button 238b cannot be actuated. In the second position, the second button 238b extends out of the housing 1484 for easy actuation, and the first button 238a is positioned substantially within or fully within the housing 1484, such that the first button 238a cannot be actuated. In the illustrated embodiment, the remote unit 1166 is ergonomically shaped such that a user could easily grasp the remote unit 1166 and actuate the first button 238a and the second button 238b.

Referring to FIGS. 9A and 14 together, for example, the shaft 869 of the remote unit 1166 can extend entirely through the housing 1484, and the first button 238a can be fixedly coupled to one side of the shaft 869, and the second button 238b can be fixedly coupled to the opposite side of the shaft 869. Accordingly, if the first button 238a is actuated (e.g., depressed), then the second button 238b would be pushed out of the housing 1484 for actuation while the first button 238a would be pressed flat against or into the housing 1484. In other embodiments, such as the embodiment shown in FIG. 14, the remote unit 1166 can include a linkage between the first button 238a and the second button 238b that allows the first button 238a and the second button 238b to be positioned along separate axes.

Referring to FIG. 14, the first button 238a can be actuated in the first position, causing aspiration of the catheter 103 (FIG. 11). For example, once the first button 238a is actuated, the remote unit 1166 can operate similarly to the remote unit 766 described in greater detail above with reference to FIG. 8A. Further, once the first button 238a is fully actuated (e.g., the remote unit 1166 is in the second position), the second button 238b can extend out of the housing 1484, and the first button 238a can be locked into a position substantially within or fully within the housing 1484 until the second button 238b is actuated. In the second position, the second button 238b can be actuated to depress the plunger assembly 222 (FIG. 2) of the pneumatic syringe 210 (FIG. 2) to direct the clot material into the filtering device 1112 or for reinfusion of blood. For example, once the second button 238b is actuated, the remote unit 1166 can operate similarly to the remote unit 766 described in greater detail above with reference to FIG. 8B.

FIG. 15 is a flow diagram of a method 1500 for removing clot material from a patient in accordance with additional embodiments of the present technology. The method 1500 is illustrated as a series of steps, process portions, or blocks 1502-1512. One or more of the blocks 1502-1512 of the method 1500 can be performed using one or more of the devices and/or systems described herein. For example, several blocks of the method 1500 are described with reference to the system 100. However, a person of ordinary skill in the art will appreciate that the system 600, system 700, the system 1000, the system 1100, and/or any of the filtering devices described herein can also be used to perform one or more blocks of the method 1500.

At block 1501, the method 1500 can include positioning a catheter of a clot treatment system near clot material within a patient. Positioning the catheter can include positioning the catheter 103 of the system 100 near clot material within a blood vessel of the patient, as described previously herein and at least with reference to FIG. 1.

At block 1504, the method 1500 can include aspirating at least a portion of the clot material into a pressure source of the clot treatment system via the catheter. Aspirating the clot material into the pressure source can include applying a vacuum stored within the first pressure source to the catheter or simultaneously generating a vacuum with the pneumatic syringe 210 and applying the vacuum to the catheter 103, as described previously herein at least with reference to FIG. 15B. In some embodiments, aspirating the clot material can include opening a first fluid control device (e.g., the fluid control device 214a of FIG. 2) of the connector (e.g., the connector 213 of FIG. 2) and closing a second fluid control device (e.g., the fluid control device 214b of FIG. 2) of the connector to direct the aspirated clot material along a first flow path (e.g., the first flow path 216a of FIG. 2) of the connector and into the first pressure source.

At block 1503, the method 1500 can include emptying aspirated material within the first pressure source into a filtering device. Emptying the aspirated material into the filtering device can include closing a first fluid control device (e.g., the first fluid control device 214a of FIG. 2) of a connector (e.g., the connector 213 of FIG. 2) and opening a second fluid control device (e.g., the second fluid control device 214b of FIG. 2) of the connector to direct the aspirated clot material along a second flow path (e.g., the second flow path 216b of FIG. 2) of the connector such that the pneumatic syringe 210 discharges or expels all, or at least a portion, of the aspirated material along the second flow path, toward and/or into the filtering device (via, e.g., the tubing section 207 of FIG. 2). When the first pressure source includes a syringe, emptying the aspirated material into the filtering device can include actuating a plunger of the syringe to empty aspirated material contained within a barrel of the syringe into the filtering device.

Block 1503 and/or the method 1500 can omit certain steps associated with disconnecting and/or reconnecting the pressure source from or to other aspects of the clot treatment system before, while, and/or after emptying the aspirated material into the filtering device. For example, emptying the aspirated material into the filtering device in block 1503 can include emptying the aspirated material without disconnecting the pressure source (block 1502) from the catheter (block 1502) and/or connecting the pressure source to the filtering device (block 1504).

At block 1504, the method 1500 can include determining whether there is additional material to aspirate from the patient. If there is additional material to aspirated from the patient (block 1504, YES), the method 1500 can return to block 1502. If not (block 1504, NO), the method 1500 can include block 1505. When returning to block 1502, the user need not connect the pressure source (block 1502) to the catheter (block 1502) and/or disconnect the pressure source from the filtering device (block 1504) for the same reasons set forth above with reference to block 1503.

At block 1505, the method 1500 can include removing blood from the filtering device. Removing blood from the filtering device can include using a syringe or other pressure source, such as the second pressure source 111, to remove blood from the filtering device, as described previously herein and at least with reference to FIGS. 1 and 6. In some embodiments, removing blood from the filtering device can include removing blood from the filtering device via an outlet conduit and/or a fluid control device. Additionally, or alternatively, removing blood from the filtering device can include drawing the blood through a filter to separate the blood from one or more other portions of the aspirated material.

At block 1506, the method 1500 can include reinfusing the filtered blood into the patient. Reinfusing the filter blood into the patient can include using the second pressure source 111 (or another pressure source, container, etc.) to reinfuse the filtered blood, as described previously herein at least with reference to FIG. 1.

The following examples are illustrative of several embodiments of the present technology:
1. A pneumatic syringe, comprising:
   a syringe barrel;
   a pneumatic barrel;
   a plunger assembly, comprising:
      a first sealing head slidably positioned within the syringe barrel;
      a second sealing head slidably positioned within the pneumatic barrel and dividing the pneumatic barrel into a first chamber and a second chamber; and
      a shaft coupling the first sealing head to the second sealing head such that the first and second sealing heads are constrained to move together;
   a control unit configured to direct a flow of pressurized fluid from a fluid source;
   a first fluid line fluidly coupling the control unit to the first chamber of the pneumatic barrel; and
   a second fluid line fluidly coupling the control unit to the second chamber of the pneumatic barrel;
   wherein the control unit is configured to move between—
      a first position in which the flow of pressurized fluid is directed to the first chamber via the first fluid line to pressurize the first chamber to drive the second sealing head proximally through the pneumatic barrel to thereby withdraw the first sealing head proximally through the syringe barrel to generate negative pressure in the syringe barrel; and
      a second position in which the flow of pressurized fluid is directed to the second chamber via the second fluid line to pressurize the second chamber to drive the second sealing head distally through the pneumatic barrel to thereby depress the first sealing head distally through the syringe barrel to generate positive pressure in the syringe barrel.
2. The pneumatic syringe of example 1 wherein the syringe barrel comprises vents configured to allow pressure from developing proximal to the first sealing head.
3. The pneumatic syringe of example 2 wherein the control unit includes an actuator configured to move the control unit from the first position to the second position when actuated.
4. The pneumatic syringe of example 3 wherein the fluid source is fluidly coupled to the control unit via a pressure regulator configured to regulate pressure within the control unit.
5. The pneumatic syringe of example 4 wherein the pneumatic syringe further includes a remote unit configured to control the control unit.
6. A pneumatic syringe, comprising:
a syringe comprising:
   a first barrel; and
   a first portion of a plunger assembly slidably positioned within the first barrel, wherein the plunger assembly is movable between a first and second position and is configured to be withdrawn through the first barrel to generate vacuum pressure in the first barrel;
a pneumatic cylinder coupled to the syringe comprising:
   a second barrel;
   a second portion of the plunger assembly slidably positioned within the second barrel; and
   two or more ports; and
a control unit fluidly coupled to the two or more ports configured to move the plunger assembly from the first position to the second position and from the second position to the first position using pressurized gas from a pressurized canister.
7. The pneumatic syringe of example 6 wherein the plunger assembly includes a first sealing head, a second sealing head, and a shaft.
8. The pneumatic syringe of example 7 wherein the first sealing head is positioned within the first barrel and the second sealing head is positioned within the second barrel.
9. The pneumatic syringe of example 8 wherein in the first position the pressurized gas pushes the second sealing head toward a distal end portion of the second barrel, generating a vacuum within the first barrel of the syringe via the first sealing head.
10. The pneumatic syringe of example 9 wherein in the second position the pressurized gas pushes the second sealing head toward a proximal portion of the second barrel, expelling contents of the syringe via the first sealing head.
11. The pneumatic syringe of example 10 wherein the two or more ports include a first port and a second port and wherein the control unit comprises a shaft configured to direct the pressurized gas through either the first port or the second port.
12. The pneumatic syringe of example 11 wherein the plunger assembly moves to the first position when the pressurized gas is directed to the first port and wherein the plunger assembly moves to the second position when the pressurized gas is directed to the second port.
13. The pneumatic syringe of any of examples 6-12 wherein the control unit includes: three or more ports fluidly coupled to the two or more ports of the pneumatic cylinder and the pressurized canister, and one or more vents.
14. A method of treating clot material within a patient, the method comprising: inserting a catheter of a clot treatment system into the patient;
actuating a pneumatic syringe to aspirate the catheter;
actuating the pneumatic syringe again to push aspirated material into a filtering device; and
repeating actuation of the pneumatic syringe until the clot material is completely removed from the patient.
15. The method of example 14 wherein actuating the pneumatic syringe includes compressing a button to change flow of pressurized gas within the pneumatic syringe.
16. The method of example 15 wherein the pneumatic syringe includes a pneumatic cylinder and a syringe and wherein actuating the pneumatic syringe includes delivering pressurized gas to the pneumatic cylinder.
17. The method of example 16 wherein repeating actuation of the pneumatic syringe includes repeatedly pressing a button.
18. The method of example 17 wherein actuating the pneumatic syringe to aspirate the catheter includes pulling the aspirated material through a one-way flow valve and wherein actuating the pneumatic syringe again to push the aspirated material into the filtering device includes pushing the aspirated material through the one-way flow valve.
19. The method of any of examples 14-18 wherein actuating the pneumatic syringe includes changing flow of pressurized gas within a control unit.
20. A system for treating clot material within a patient comprising:
a catheter configured to be inserted into the patient;
a pressure source including:
   a syringe coupled to
   a pneumatic cylinder;
   a plunger assembly slidably positioned within the syringe and the pneumatic cylinder, movable between a first and second position, and configured to be withdrawn through the syringe to generate vacuum pressure in the syringe; and
   a control unit coupled to the pneumatic cylinder configured to move the plunger assembly from the first position to the second position and from the second position to the first position;
a filtering device; and
a tubing assembly fluidly coupling the catheter, the pressure source, and the filtering device.
21. The system of example 20 wherein the system further comprises a second pressure source configured to return filtered blood to the patient through the tubing assembly.
22. The system of example 21 wherein moving the plunger assembly from the first position to the second position aspirates the catheter.
23. The system of example 22 wherein moving the plunger assembly from the second position to the first position pushes aspirated material to and/or through the filtering device.
24. The system of any of examples 20-23 wherein the control unit uses pressurized gas to move the plunger assembly between the first and second positions.

All numeric values are herein assumed to be modified by the term about whether or not explicitly indicated. The term about, in the context of numeric values, generally refers to a range of numbers that one of skill in the art would consider equivalent to the recited value (e.g., having the same function and/or result). For example, the term about can refer to the stated value plus or minus ten percent. For example, the use of the term about 100 can refer to a range of from 90 to 110, inclusive. In instances in which the context requires otherwise and/or relative terminology is used in reference to something that does not include, or is not related to, a numerical value, the terms are given their ordinary meaning to one skilled in the art.

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A pneumatic syringe, comprising:
a syringe barrel;
a pneumatic barrel;
a plunger assembly, comprising:
a first sealing head slidably positioned within the syringe barrel;
a second sealing head slidably positioned within the pneumatic barrel and dividing the pneumatic barrel into a first chamber and a second chamber; and
a shaft coupling the first sealing head to the second sealing head such that the first and second sealing heads are constrained to move together;
a control unit configured to direct a flow of pressurized fluid from a fluid source;
a first fluid line fluidly coupling the control unit to the first chamber of the pneumatic barrel; and
a second fluid line fluidly coupling the control unit to the second chamber of the pneumatic barrel;
wherein the control unit is configured to move between—
a first position in which the flow of pressurized fluid is directed to the first chamber via the first fluid line to pressurize the first chamber to drive the second sealing head proximally through the pneumatic barrel to thereby withdraw the first sealing head proximally through the syringe barrel to generate negative pressure in the syringe barrel; and
a second position in which the flow of pressurized fluid is directed to the second chamber via the second fluid line to pressurize the second chamber to drive the second sealing head distally through the pneumatic barrel to thereby depress the first sealing head distally through the syringe barrel to generate positive pressure in the syringe barrel.

2. The pneumatic syringe of claim 1 wherein the syringe barrel comprises vents configured to allow pressure from developing proximal to the first sealing head.

3. The pneumatic syringe of claim 1 wherein the control unit includes an actuator configured to move the control unit from the first position to the second position when actuated.

4. The pneumatic syringe of claim 1 wherein the fluid source is fluidly coupled to the control unit via a pressure regulator configured to regulate pressure within the control unit.

5. A pneumatic syringe, comprising:
a syringe comprising:
a first barrel; and
a first portion of a plunger assembly slidably positioned within the first barrel, wherein the plunger assembly is movable between a first and second position and is configured to be withdrawn through the first barrel to generate vacuum pressure in the first barrel;
a pneumatic cylinder coupled to the syringe comprising:
a second barrel;
a second portion of the plunger assembly slidably positioned within the second barrel; and
two or more ports; and
a control unit fluidly coupled to the two or more ports configured to move the plunger assembly from the first position to the second position and from the second position to the first position using pressurized gas from a pressurized canister.

6. The pneumatic syringe of claim 5 wherein the plunger assembly includes a first sealing head positioned within the first barrel, a second sealing head positioned within the second barrel, and a shaft connecting the first sealing head and the second sealing head.

7. The pneumatic syringe of claim 6 wherein in the first position the pressurized gas pushes the second sealing head toward a distal end portion of the second barrel, generating a vacuum within the first barrel of the syringe via the first sealing head.

8. The pneumatic syringe of claim 6 wherein in the second position the pressurized gas pushes the second sealing head toward a proximal portion of the second barrel, expelling contents of the syringe via the first sealing head.

9. The pneumatic syringe of claim 5 wherein the two or more ports include a first port and a second port and wherein the control unit comprises a shaft configured to direct the pressurized gas through either the first port or the second port.

10. The pneumatic syringe of claim 9 wherein the plunger assembly moves to the first position when the pressurized gas is directed to the first port and wherein the plunger assembly moves to the second position when the pressurized gas is directed to the second port.

11. The pneumatic syringe of claim 5 wherein the control unit includes:
three or more ports fluidly coupled to the two or more ports of the pneumatic cylinder and the pressurized canister, and
one or more vents.

12. A method of treating clot material within a patient, the method comprising:
inserting a catheter of a clot treatment system into the patient;
actuating a pneumatic syringe to aspirate the catheter;
actuating the pneumatic syringe again to push aspirated material into a filtering device; and
repeating actuation of the pneumatic syringe until the clot material is completely removed from the patient.

13. The method of claim 12 wherein the pneumatic syringe includes a pneumatic cylinder and a syringe, and wherein actuating the pneumatic syringe includes delivering pressurized gas to the pneumatic cylinder.

14. The method of claim 12 wherein repeating actuation of the pneumatic syringe includes repeatedly pressing a button.

15. The method of claim 12 wherein actuating the pneumatic syringe to aspirate the catheter includes pulling the aspirated material through a one-way flow valve, and wherein actuating the pneumatic syringe again to push the aspirated material into the filtering device includes pushing the aspirated material through the one-way flow valve.

16. The method of claim 12 wherein actuating the pneumatic syringe includes changing flow of pressurized gas within a control unit.

17. A system for treating clot material within a patient comprising:
a catheter configured to be inserted into the patient;
a pressure source including:
a syringe coupled to
a pneumatic cylinder;
a plunger assembly slidably positioned within the syringe and the pneumatic cylinder, movable between a first and second position, and configured to be withdrawn through the syringe to generate vacuum pressure in the syringe; and
a control unit coupled to the pneumatic cylinder configured to move the plunger assembly from the first position to the second position and from the second position to the first position;
a filtering device; and
a tubing assembly fluidly coupling the catheter, the pressure source, and the filtering device.

18. The system of claim 17 wherein the system further comprises a second pressure source configured to return filtered blood to the patient through the tubing assembly.

19. The system of claim 17 wherein moving the plunger assembly from the first position to the second position aspirates the catheter.

20. The system of claim 17 wherein moving the plunger assembly from the second position to the first position pushes aspirated material to and/or through the filtering device.
